(19) United States
(12) Patent Application Publication
(10) Pub. No.: US 2024/0287456 A1
(43) Pub. Date: Aug. 29, 2024

(54) A METHOD FOR PRODUCING ANTIGEN-SPECIFIC T CELLS

(71) Applicant: Achilles Therapeutics UK Limited, London (GB)

(72) Inventors: Katy Rebecca Newton, London (GB); Eleni Kotsiou, London (GB); Joe Robinson, London (GB); Sergio Quezada, London (GB); Henrieta Fraser, London (GB); Sarah Thirkell, London (GB)

(73) Assignee: Achilles Therapeutics UK Limited, London (GB)

(21) Appl. No.: 18/572,495

(22) PCT Filed: Jun. 21, 2022
(86) PCT No.: PCT/GB2022/051581

§ 371 Date: Dec. 20, 2023

(30) Foreign Application Priority Data
Jun. 22, 2021 (GR) 20210100409

(51) Int. Cl. C12N 5/0783; A61K 35/17; A61K 39/00

(52) U.S. Cl. CPC C12N 5/0636; A61K 35/17; A61K 39/4611; A61K 39/464401; C12N 2501/2302; C12N 2501/2315; C12N 2501/2321; C12N 2501/24; C12N 2501/51; C12N 2501/515; C12N 2501/53; C12N 2502/1107; C12N 2502/1121

(57) ABSTRACT

The present invention relates to a method for producing antigen-specific T cells and their use in a method for the treatment or prevention of cancer.

### FIELD OF THE INVENTION

The present invention relates to a method for producing T cells, such as antigen-specific T cells, and their use in a method for the treatment or prevention of cancer.

### BACKGROUND TO THE INVENTION

Cancer immunotherapy uses the body's own immune system to target, control and eliminate cancer. One type of cancer immunotherapy is adoptive T cell therapy, whereby T cells are isolated or engineered, expanded ex vivo, and transferred back to patients. The T cells are either derived from the patient themselves (autologous) or from a donor (allogeneic).

High numbers of T cells may be required for an effective T cell therapy, in order to provide a sufficient dose of the T cells to a patient. The generation of large numbers or high doses of T cells has previously been investigated in the art. However, some T cell therapies require the T cell population to contain increased numbers of antigen-specific T cells, in particular. The T cells with antigen-specificity should be of a functional fitness that allows their effective use in a T cell therapy. The provision of T cell therapies which can deliver high doses of T cells with increased antigen-specificity, as well as functional fitness, is therefore highly desirable.

Previous methods for generating T cells for use in T cell therapies do not increase the dose of antigen-specific T cells in particular, rather they are directed to non-specific expansion of T cells. Such T cells are not suitable for T cells therapies requiring the T cells to have specificity towards particular antigens. As such, there is a need in the art for alternative and improved methods for producing populations of functionally fit T cells that have specificity towards particular antigens.

### BRIEF DESCRIPTION OF THE INVENTION

The present inventors have developed a new method for antigen-specific expansion of T cells. The invention provides a method for providing a population of T cells containing higher numbers of antigen-specific T cells than has previously been achieved, whilst maintaining T cell fitness and functionality. The T cell expansion methods of the invention facilitate production of greater numbers of antigen-specific T cells within a T cell population.

Methods according to the present invention provide an improvement over previous methods, in that a population containing high numbers of T cells may be produced, and wherein said population contains an increased number or proportion of antigen-specific T cells, and furthermore wherein the T cells in the population are functionally fit. Functional fitness of the T cells can be determined by assessment of various markers as described below.

The method of the invention comprises an antigen-specific T cell expansion step, followed by a non-specific expansion step. The non-specific expansion step functions to boost the number of antigen-specific T cells, and may be referred to herein as a “boost expansion step”.

The method of the invention may also comprise an optional step of non-specific expansion of the T cells before the antigen-specific expansion step. This may be referred to as a “pre-expansion step”.

In one aspect the invention provides a method for producing a population of T cells which comprises antigen-specific T cells, wherein said method comprises an antigen-specific T cell expansion step followed by a non-specific T cell expansion step (boost expansion step). Optionally, the antigen-specific expansion step may also be preceded by a non-specific T cell pre-expansion step as described herein.

The antigen-specific expansion step as described herein may increase the number or proportion of T cells specific to a particular antigen within the T cell population.

In one aspect the invention provides a method for producing a population of T cells which comprises antigen-specific T cells, wherein said method comprises the steps of:
a) an antigen-specific expansion step comprising co-culturing isolated T cells with antigen-presenting cells that have been loaded with antigen, wherein said T cells and antigen-presenting cells are co-cultured in the presence of IL-2; and
b) a non-specific boost expansion step comprising culturing the T cells produced in step (a) in the presence of anti-CD3 antibodies and/or anti-CD28 antibodies and/or anti-CD2 antibodies and/or IL-2.

The method may further comprise a non-specific pre-expansion step prior to the antigen-specific expansion step, comprising culturing isolated T cells in the presence of IL-2 and IL-21.

The pre-expansion step may further comprise culturing the T cells in the presence of anti-CD3 antibodies, anti-CD28 antibodies, anti-CD2 antibodies and/or IFNγ.

In one aspect the pre-expansion step may comprise culturing the T cells in the presence of IL-2, IL-15, IL-21, anti-CD3 antibodies, anti-CD28 antibodies and anti-CD2 antibodies.

In one aspect the antigen-specific expansion step comprises co-culturing the T cells and antigen-presenting cells in the presence of IL-2 and IL-15.

The antigen-specific expansion step may be carried out in cell culture medium that comprises a serum replacement. In one aspect the serum replacement may comprise platelet lysate.

The method according to the invention may further comprise a non-specific “pre-expansion” step which involves initially expanding the isolated T cells in a non-antigen-specific way.

In one aspect the pre-expansion step comprises culturing the T cells in the presence of IL-2 and IL-21, IL-2 and IL-15, or IL-2, IL-15 and IL-21. The pre-expansion step may comprise culturing T cells in the presence of platelet lysate. The pre-expansion step may further comprise culturing T cells in the presence of anti-CD3, anti-CD3/28 or anti-CD3/28/2 antibodies and/or interferon-gamma to further increase the number of T cells produced.

In one aspect the pre-expansion step is of a duration of about 7 to about 21 days, for example about 14 to 16 days.

In one aspect the T cells have been isolated from a tumour of a subject with cancer. In one aspect the isolated T cells are tumour infiltrating lymphocytes (TIL).

The “antigen-specific expansion” step may occur subsequent to the pre-expansion step.

The antigen-presenting cells referred to in the antigen specific-expansion step are preferably dendritic cells, for example autologous dendritic cells. Dendritic cells may be produced from monocytes obtained from a blood sample, to provide monocyte-derived dendritic cells (MoDCs). In one aspect, the antigen presenting cells are autologous MoDCs, which are produced from the patient's own blood sample.

In one aspect the IL-2 in the antigen-specific expansion step may be used at a concentration of 500 U/ml or below.

In one aspect the antigen-specific expansion step has a duration of about 7 to about 21 days, for example about 10 days or about 17 days.

The antigen-specific expansion step may result in an increased number of total T cells in the population, and preferably an increased number or proportion of antigen-specific T cells, compared with the isolated T cells of the pre-expansion step.

In one aspect the non-specific boost expansion step comprises culturing T cells from the antigen-specific expansion step in the presence of one or more of:
(i) anti-CD3 antibody;
(ii) anti-CD28 antibody; and
(iii) IL-2.

In one aspect the non-specific boost expansion step may have a duration of about 3 days to about 21 days, for example about 7 days or about 17 days.

The boost expansion step may result in an increased number of total T cells in the population, and preferably an increased number of antigen-specific T cells, compared with a starting population (for example, the isolated T cells of the pre-expansion step and/or the population of cells in the antigen-specific expansion step).

In one aspect the pre-expansion and/or antigen-specific expansion steps further comprise culturing the T cells in the presence of IL-15.

In one aspect the method of the invention comprises:
a) an antigen-specific expansion step comprising co-culturing isolated T cells with antigen presenting cells that have been loaded with antigen, wherein said T cells and antigen presenting cells are co-cultured in the presence of IL-2 and IL-21; and
b) a non-specific boost expansion step comprising culturing the cells produced in step a) in the presence of anti-CD3 antibodies and/or anti-CD28 antibodies and/or IL-2.

In one aspect the method of the invention comprises:
a) a non-specific pre-expansion step comprising culturing isolated T cells in the presence of IL-2 and IL-21;
b) an antigen-specific expansion step comprising co-culturing said T cells with antigen presenting cells that have been loaded with antigen, wherein said T cells and antigen presenting cells are co-cultured in the presence of IL-2; and
c) a non-specific boost expansion step comprising culturing the cells produced in step b) in the presence of anti-CD3 antibodies and/or anti-CD28 antibodies and/or IL-2.

In one aspect the method of the invention comprises:
a) an antigen-specific expansion step comprising co-culturing said T cells with antigen presenting cells that have been loaded with antigen, wherein said T cells and antigen presenting cells are co-cultured in the presence of IL-2, IL- and IL-21; and
b) a non-specific boost expansion step comprising culturing the cells produced in step a) in the presence of anti-CD3 antibodies and/or anti-CD28 antibodies and/or IL-2.

In one aspect the method of the invention comprises:
a) a non-specific pre-expansion step comprising culturing isolated T cells in the presence of IL-2, IL-15 and IL-21;
b) an antigen-specific expansion step comprising co-culturing said T cells with antigen presenting cells that have been loaded with antigen, wherein said T cells and antigen presenting cells are co-cultured in the presence of IL-2 and IL-15; and
c) a non-specific boost expansion step comprising culturing the cells produced in step b) in the presence of anti-CD3 antibodies and/or anti-CD28 antibodies and/or IL-2.

The methods according to the invention may advantageously provide a population of T cells, wherein said T cells display functional markers, for example production of IFNγ and expression of CD25 and/or CD27. Said T cells may also express decreased amounts of the exhaustion marker CD57.

The methods of the invention may advantageously provide a population of T cells which have a more even balance of CD4+ and CD8+ T cells. For example, the methods of the invention as described herein may result in a population of T cells which contains more CD8+ cells than previous methods. The T cell population may therefore be more balanced for CD4+/CD8+ T cells than a T cell population achieved by previous methods. In one aspect the T cell population comprises at least about 20%, 30%, 50%, 70% or 80% or more CD8+ T cells.

The invention encompasses a population of T cells produced by any of the methods as described herein. The population of T cells may have increased numbers of T cells than a population of T cells isolated from a subject. The T cell population may have an increased proportion of T cells specific for one or more particular antigens. The T cell population may be enriched with T cells specific for one or more particular antigens.

The methods according to the invention may facilitate production of a T cell population comprising at least about 10×10⁶ antigen-specific T cells. A T cell population produced according to the invention may provide a dose of at least about 10×10⁶ antigen-specific T cells to a subject. In one aspect the T cell population may comprise between about 10×10⁶ and about 1×10¹⁰ antigen-specific T cells, for example between about 1×10⁸ and about 1×10⁹, such as about 2×10⁸ antigen-specific T cells.

A T cell population produced by the method of the invention may comprise T cells with the CD3+/CD56− phenotype.

T cells produced according to the present invention may upregulate IL-2 (CD25) expression upon restimulation with an antigen. In one aspect, the same antigen is used for both antigen-specific expansion and for the restimulation.

The methods according to the invention may produce a T cell population comprising predominantly effector memory T cells with a phenotype associated with the cytotoxic (killer) phenotype.

The T cell population produced according to the method of the invention may be used in medicine as a T cell therapy, preferably in the treatment or prevention of cancer in a subject.

The methods according to the present invention may be carried out in vitro or ex vivo.

### DESCRIPTION OF THE FIGURES

FIG. 1: Fold expansion of T cells (CD3+CD56−) determined by cell counts at Day 0 and Day 17 of the specific/non-specific expansion period

FIG. 2: Total T cell dose (number of T cells (CD3+CD56−), scaled to tumour weight

FIG. 3: cNeT dose/reactivity, measured by IFNγ/TNFα positive cells, scaled to tumour weight

FIG. 4: Fold increase in cNeT dose.

FIG. 5: Memory phenotype of CD4+ and CD8+ T cells for each process

FIG. 6: Proportion of CD8+ and CD4+ T cells with marker expression (median)

FIG. 7: Levels of CD25 expression produced when cNeT restimulated with clonal neoantigen peptides.

FIG. 8: Data generated from 8 further cancer patients comparing reactive cells dose for each process.

FIG. 9: Fold change in TIL yield for Gen 2.8.1 process compared to Gen 2.6 process.

FIG. 10: Fold change in cNeT dose for Gen 2.8.1 process compared to Gen 2.6 process.

FIG. 11: Fold change in TIL yield for Gen 2.8.2 process compared to Gen 2.8.1 process.

FIG. 12: Fold change in T cell expansion for Gen 2.6 B cell and Gen 2.8.1 B cell compared to Gen 2.6 and Gen 2.8.1.

FIG. 13: Fold change in proportion of cNeT for Gen 2.8.1 B cell and Gen 2.6 B cell compared to Gen 2.8.1 and Gen 2.6.

FIG. 14: Proportion of CD4+ and CD8+ T cells in a Gen 2.6 product compared to the corresponding Gen 2.6 B cell product.

### DETAILED DESCRIPTION OF THE INVENTION

The present invention provides a method for producing a population of T cells, wherein said population comprises antigen-specific T cells. Advantageously, the methods of the invention as described herein facilitate production of a T cell population which contains an increased number or proportion of antigen-specific T cells, which are functionally fit and suitable for use in a T cell therapy.

The method according to the invention comprises an antigen-specific expansion step wherein the T cells are co-cultured with antigen presenting cells which have been loaded with one or more antigens, followed by a non-specific expansion step which boosts the number of T cells. The number of T cells specific for (or reactive to) said antigen(s) is increased in the specific expansion step. The proportion or percentage of antigen-specific T cells in the population of T cells may increase.

In the context of the present invention, the term “expansion” or “expanding” means increasing the number of T cells by inducing their proliferation. T cells may be expanded by ex vivo culture in conditions which provide mitogenic stimuli for T cells.

By “antigen-specific expansion step” is meant a step of increasing the number of T cells in the presence of antigen. The presence of antigen leads to an increase in, or expansion of, T cells with specificity to said antigen within the overall population. The aim of this step is to preferentially or selectively expand T cells that bind and respond to one or more antigens. As a consequence, the antigen-specific expansion step typically employs lower concentrations of IL-2 (such as 500 U/ml or lower) compared to non-specific expansion steps, in order to minimise any non-specific expansion of the T cells. An antigen-specific expansion step increases the proportion or percentage of T cells specific to said antigen within the overall population of T cells, i.e. compared to the proportion or percentage of T cells not specific to said antigen.

In one aspect of the invention, the antigen-specific expansion step comprises co-culturing T cells with antigen presenting cells (APCs) that have been loaded with antigen, or peptide derived from antigen, in the presence of IL-2. When a T cell recognises its cognate antigen presented by the APC, this provides one of the required signals, together with cytokine stimulation, that enables the T cell to expand (i.e. proliferate). This process allows selective expansion of the T cells of interest.

By “non-specific boost expansion step” is meant a step of increasing the number of T cells in the absence of antigen. The lack of antigen leads to an overall (or general) increase in, or expansion of, T cells in the population irrespective of their antigen-specificity.

The method according to the invention may further comprise a non-specific pre-expansion step wherein isolated T cells, for example in the form of tumour single-cell suspensions or tumour fragments, are cultured in vitro in the presence of IL-2, and optionally with one or more of: IL-15, IL-21, anti-CD3 antibodies, anti-CD28 antibodies and/or anti-CD2 antibodies.

### Isolated T Cells

The T cell population may be generated from T cells in a sample isolated from a subject with a tumour. The sample may be taken from a tumour, peripheral blood (e.g. peripheral blood mononuclear cells or PBMC), bone marrow, lymph node tissue, cord blood, thymus tissue, tissue from a site of infection, ascites, pleural effusion, spleen tissue or from other tissues of the subject.

T cells can be obtained from a sample of blood collected from a subject using any number of techniques known to the skilled person. For example, density gradient separation techniques, such as FICOLL™ separation, and/or apheresis, such as leukapheresis, may be employed. Additional methods of isolating T cells for a T cell therapy are disclosed in U.S. Patent Publication No. 2013/0287748, which is herein incorporated by reference in its entirety.

In a particular embodiment, the T cell population is generated from a sample from the tumour. In other words, the T cell population is isolated from a sample obtained from the tumour of a patient to be treated. Such T cells are referred to herein as ‘tumour infiltrating lymphocytes’ (TIL). TIL are T cells that have infiltrated tumour tissue.

The isolated T cells in the method according to the invention may be TIL.

Isolation of biopsies and samples from tumours is common practice in the art and may be performed according to any suitable method and such methods will be known to one skilled in the art.

The tumour may be a solid tumour or a non-solid tumour.

T cells may be isolated using methods which are well known in the art. For example, TIL may be isolated by culturing resected tumour fragments or tumour single-cell suspensions in medium containing IL-2. T cells may be purified from single cell suspensions generated from samples on the basis of expression of CD3, CD4 or CD8. T cells may be enriched from samples by passage through a density gradient.

### Antigen Presenting Cells

An antigen-presenting cell (APC) or accessory cell is a cell that displays antigen complexed with major histocompatibility complexes (MHCs) on their surfaces; this process is known as antigen presentation. T cells may recognize these complexes using their T cell receptors (TCRs).

In one aspect, the antigen presenting cell is a dendritic cell. The dendritic cell (DC) may be derived from monocytes isolated from blood to produce monocyte-derived dendritic cells (MoDCs). In one aspect, the DCs are produced from a blood sample obtained from the patient, to produce autologous DCs. In a preferred aspect, the DCs are autologous MoDCs. Standard methods in the art may be used to produce dendritic cells from isolated monocytes. For example, a protocol for obtaining PBMC-derived DCs is described in Leko et al. (J. Immunol. 2019, 202: 3458-3467). Further, DC purification/isolation kits are commercially available, such as e.g. EasySep™ DC enrichment kits from StemCell™ Technologies. In addition, CD14 Microbeads and associated protocols are available from Miltenyi Biotech (available at https://www.miltenyibiotec.com/GB-en/products/cd14-microbeads-human.html#130-050-201).

In one aspect, the antigen presenting cell is a B cell. In one aspect, the B cell is expanded from blood, for example a blood sample obtained from the patient. In one aspect, the B cells are expanded from CD19+ cells isolated from a blood sample. Any suitable method may be used to isolate CD19+, such as positive or negative selection using immunomagnetic particles coated with anti-CD19 antibodies. CD19 purification/isolation reagents and kits are commercially available, such as e.g. CD19 MicroBeads or B Cell Isolation Kit II, human (Miltenyi Biotec) and EasySep™ Human CD19 Positive Selection Kit (StemCell™ Technologies). Another approach is to use positive selection for CD20 or CD22, for example using CD20 or CD22 MicroBeads (Miltenyi Biotec).

Standard methods known in the art may be used to produce B cells from isolated CD19+ monocytes or directly from blood samples or PBMCs. For example, a protocol for B cell expansion is described in Kotsiou et al. (Blood 2016, 128:72-81) using CD40L, F(ab′)2 fragment goat anti-IgA+IgG+IgM, CpG and IL-4. Another typical method is culture with CD40L expressing feeder cells as taught by Su et al (J Immunol 2016, 197:4163-4176). B cell expansion kits are commercially available, such as e.g. ImmunoCult™ Human B Cell Expansion Kit from StemCell™ Technologies and B Cell Expansion Kit, Human from Miltenyi Biotec.

In one aspect, isolated CD19+ cells are cultured with IL-4, CD40L and CpG to expand B cells.

In one aspect, the B cell expansion medium comprises IL-4 at a concentration of about 10 to 100 ng/ml, for example about 25 to 75 ng/ml. In some embodiments, the B cell expansion medium comprises about 50 ng/ml of IL-4. In an embodiment, the B cell expansion medium comprises about 10 ng/mL, about 25 ng/ml, about 30 ng/ml, about ng/ml, about 40 ng/ml, about 45 ng/ml, about 50 ng/ml, about 55 ng/ml, about 60 ng/ml, about 70 ng/ml, about 80 ng/ml, about 90 ng/ml or about 100 ng/ml of IL-4. In an embodiment, the B cell expansion medium comprises between 10 ng/ml and ng/ml, between 20 ng/ml and 30 ng/ml, between 30 ng/ml and 40 ng/ml, between ng/ml and 50 ng/ml, or between 50 ng/mL and 100 ng/ml of IL-4.

In one aspect, the B cell expansion medium comprises CD40L at a concentration of about 0.5 to about 50 IU/mL, for example about 0.5 to about 10, 12, 15, or 20 IU/mL, or alternatively about 2.5 to 25 IU/ml. In one aspect the CD40L is present at a concentration of about 40 IU/mL, about 35 IU/mL, about 30 IU/mL, about 25 IU/mL, about 20 IU/mL, about 15 IU/mL, about 12 IU/mL, about 10 IU/mL, about 5 IU/mL, about 4 IU/mL, about 3 IU/mL, about 2 IU/mL, about 1 IU/mL or about 0.5 IU/mL. In one aspect the CD40L is present at a concentration of about 12 IU/mL.

In one aspect, the B cell expansion medium comprises CpG at a concentration of about 0.1 to about 10 μg/mL, for example about 0.5 to about 3, 4, 5, or 6 μg/mL, or alternatively about 4 to 5 μg/mL. In one aspect the CD40L is present at a concentration of about 10 μg/mL, about 9 μg/mL, about 8 μg/mL, about 7 μg/mL, about 6 μg/mL, about 5 μg/mL, about 4.5 μg/mL, about 4 μg/mL, about 3 μg/mL, about 2 μg/mL, about 1 μg/mL or about 0.5 μg/mL. In one aspect the CD40L is present at a concentration of about 4.6 μg/ml.

The antigen presenting cells may be used at a ratio of from about 2:1 to about 1:100, such as about 1:1, 1:2, 1:3, 1:4, 1:5, 1:10, 1:20, 1:50 or 1:75 APCs to T cells.

In one aspect, the antigen presenting cells have been loaded with antigen. Loading of antigen may be achieved by methods known in the art. For example, antigen may be loaded by pulsing the antigen presenting cells (APCs) with peptide or by genetic modification. In the context of the present invention, the term “antigen” refers to one or more antigens.

Methods for loading APCs with antigens by pulsing the APCs are known in the art. For example, a protocol for loading APCs by pulsing with peptides comprising an identified mutation is described in Leko et al. (J Immunol. 2019, 202: 3458-3467).

The APCs may be loaded with antigens in the form of peptides containing one or more identified mutations as single stimulants or as pools of stimulating peptides, such as e.g. peptides comprising mutations identified as neoantigens. For example, Leko et al. describes a protocol comprising loading APCs with antigens by incubating the APCs with pools of up to 12 individual peptides each comprising an identified point mutation flanked on both sides by 12 wild type amino acids.

In one aspect, immature dendritic cells are loaded with peptide and then matured. In another aspect, mature dendritic cells are loaded with peptide. In yet another aspect, the dendritic cells are loaded with peptide twice, both when immature and mature.

Alternatively, methods for loading APCs with antigens by modifying the APCs to express the antigen are known in the art. For example, the APCs may be modified to express an antigen sequence by transfecting the APCs with mRNA encoding the antigen sequence. The mRNA encoding the antigen sequence may be in the form of a minigene or tandem minigene. The APCs may transfected with mRNA encoding peptides comprising identified mutations as constructs or as constructs encoding for multiple such peptides. For example, Leko et al. describes a protocol comprising loading APCs with antigens by electroporating the APCs with tandem minigene RNA comprising up to 12 minigenes, each comprising the coding sequence for a mutated amino acid flanked bilaterally by a sequence encoding 12 wild type amino acids.

In one aspect the antigen presenting cell is a cell capable of presenting the relevant peptide, for example in the correct HLA context. Such a cell may be an autologous cell expressing an autologous HLA molecule, or a non-autologous cell expressing an array of matched HLAs. In one aspect, the artificial antigen presenting cell is irradiated.

The term “peptide” is used in the normal sense to mean a series of residues, typically L-amino acids, connected one to the other typically by peptide bonds between the α-amino and carboxyl groups of adjacent amino acids. The term includes modified peptides and synthetic peptide analogues.

The peptide may be made using chemical methods (Peptide Chemistry, A practical Textbook. Mikos Bodansky, Springer-Verlag, Berlin.). For example, peptides can be synthesized by solid phase techniques (Roberge J Y eta/(1995) Science 269: 202-204), cleaved from the resin, and purified by preparative high performance liquid chromatography (e.g., Creighton (1983) Proteins Structures And Molecular Principles, W H Freeman and Co, New York NY). Automated synthesis may be achieved, for example, using the ABI 43 1 A Peptide Synthesizer (Perkin Elmer) in accordance with the instructions provided by the manufacturer.

The peptide may alternatively be made by recombinant means, or by cleavage from the polypeptide which is or comprises the antigen. The composition of a peptide may be confirmed by amino acid analysis or sequencing (e.g., the Edman degradation procedure).

As is well known in the art, antigens are presented to T cells in the context of antigen-derived peptides bound by major histocompatibility molecules (MHC).

Methods of predicting whether a peptide is likely to bind to a particular MHC molecule, and hence function as an antigen, are known in the art. For example, as explained below, MHC binding of peptides may be predicted using the netMHC (Lundegaard et al.) and netMHCpan (Jurtz et al.) algorithms. Thus, APCs may be loaded with peptides that are predicted using any such method as likely to be presented by one or more MHC molecules of relevance. Instead or in addition to this, APCs may be loaded with antigen using a plurality of candidate peptides each comprising a mutation of interest and differing from each other by the location of the mutation of interest in the peptide.

MHC class I proteins form a functional receptor on most nucleated cells of the body. There are 3 major MHC class I genes in HLA: HLA-A, HLA-B, HLA-C and three minor genes HLA-E, HLA-F and HLA-G. β2-microglobulin binds with major and minor gene subunits to produce a heterodimer.

Peptides that bind to MHC class I molecules are typically 7 to 13, more usually 8 to 11 amino acids in length. The binding of the peptide is stabilised at its two ends by contacts between atoms in the main chain of the peptide and invariant sites in the peptide-binding groove of all MHC class I molecules. There are invariant sites at both ends of the groove which bind the amino and carboxy termini of the peptide. Variations in peptide length are accommodated by a kinking in the peptide backbone, often at proline or glycine residues that allow the required flexibility.

There are 3 major and 2 minor MHC class II proteins encoded by the HLA locus. The genes of the class II combine to form heterodimeric (αβ) protein receptors that are typically expressed on the surface of antigen-presenting cells.

Peptides which bind to MHC class II molecules are typically between 8 and 20 amino acids in length, more usually between 10 and 17 amino acids in length and can be longer (for example up to 40 amino acids). These peptides lie in an extended conformation along the MHC II peptide-binding groove which (unlike the MHC class I peptide-binding groove) is open at both ends. The peptide is held in place mainly by main-chain atom contacts with conserved residues that line the peptide-binding groove.

The peptide may comprise a mutation (e.g. a non-silent amino acid substitution encoded by a SNV) at any residue position within the peptide. By way of example, a peptide which is capable of binding to an MHC class I molecule is typically 7 to 13 amino acids in length. As such, the amino acid substitution may be present at position 1, 2, 3, 4, 5, 6, 7, 8, 9, 10, 11, 12 or 13 in a peptide comprising thirteen amino acids.

In one aspect, longer peptides, for example peptides that are 27, 28, 29, 30 or 31 amino acids long, may be used to stimulate both CD4+ and CD8+ cells. The mutation may be at any position in the peptide. In one aspect, the mutation is at or near the centre of the peptide, e.g. at position 12, 13, 14, 15 or 16.

Any suitable number of antigens may be used in the antigen-specific expansion step, for example from 10 to 300 antigens, such as 25 to 250, 50 to 200, 70 to 185, or 100 to 150 antigens, such as about 10, 20, 50, 75, 100, 125, 150, 175, 200 or 250 antigens.

### Cytokines

According to the methods of the invention, the T cells may be cultured with cytokines as described herein.

The term “IL-2” refers to the T cell growth factor known as interleukin-2 and includes all forms of IL-2 including human and mammalian forms, conservative amino acid substitutions, glycoforms, biosimilars and variants thereof. For example, the term IL-2 encompasses human recombinant forms of IL-2 such as Aldesleukin (trade name PROLEUKIN®). Aldesleukin (des-alanyl-I, serine-125 human IL-2) is a nonglycosylated human recombinant form of IL-2 with a molecular weight of approximately 15 kDa. The term IL-2 also encompasses pegylated forms of IL-2, as described in WO 2012/065086.

In one aspect, in the non-specific pre-expansion step IL-2 is present at a concentration of about 1,000 to about 10,000 IU/mL. For example, IL-2 may be present at a concentration of about 4,000 to about 8,000 IU/mL, e.g. about 5,000 IU/mL to about 7,000 IU/mL, preferably about 6,000 IU/mL. In the non-specific pre-expansion step IL-2 may be used at a concentration of about 1,000, 2,000, 3,000, 4,000, 5,000, 6,000, 7,000, 8,000, 9,000 or 10,000 IU/mL.

The concentration of IL-2 used in the antigen-specific expansion step may be described as “lower” or “reduced”, for example in comparison to the concentration of IL-2 used in the non-specific pre-expansion or boost expansion steps. The lower concentration of IL-2 is used to promote selective expansion of the T cells in response to antigen and reduce non-specific expansion.

In a preferred aspect, in the antigen-specific expansion step IL-2 is present at a concentration of about 10 to 500 IU/mL, for example about 50 IU/ml to 250 IU/ml, preferably about 100 IU/ml. In the antigen-specific expansion step IL-2 may be used at a concentration of about 50, 75, 100, 150, 250 or 500 IU/mL.

In one aspect, in the non-specific boost and/or pre-expansion step IL-2 is present at a concentration of about 100 to 10,000 IU/mL. For example, IL-2 may be present at a concentration of about 500 to about 6,000 IU/mL, e.g. about 1,000 IU/mL to about 5,000 IU/mL, or about 3,500 to about 4,500 IU/mL, preferably about 4,000 IU/mL. In the non-specific boost expansion step IL-2 may be used at a concentration of about 500, 1,000, 2,000, 3,000, 4,000, 5,000, 6,000, 7,000, 8,000, 9,000 or 10,000 IU/mL.

The term “IL-15” refers to the immunomodulatory cytokine interleukin-15 and includes all forms of IL-15 including human and mammalian forms, conservative amino acid substitutions, glycoforms, biosimilars and variants thereof. For example, the term IL-15 encompasses human recombinant forms of IL-15.

In one aspect, the IL-15 is present at a concentration of about 10 to 1600 IU/mL, for example about 80 to 800 IU/mL. In one aspect the IL-15 is present at a concentration of about 500 IU/mL, about 400 IU/mL, about 300 IU/mL, about 200 IU/mL, about 180 IU/mL, about 160 IU/mL, about 140 IU/mL, about 120 IU/mL, or about 100 IU/mL. In one aspect the IL-15 is present at a concentration of about 100 IU/mL to about 500 IU/mL. In another aspect the IL-15 is present at a concentration of about 100 to 400 IU/ml, or about 100 to 300 IU/mL, preferably about 200 IU/mL, more preferably 160 IU/ml.

The term “IL-21” refers to the immunomodulatory cytokine interleukin-21 and includes all forms of IL-21 including human and mammalian forms, conservative amino acid substitutions, glycoforms, biosimilars and variants thereof. For example, the term IL-21 encompasses human recombinant forms of IL-21.

In one aspect, the IL-21 is present at a concentration of about 0.5 to about 50 IU/mL, for example about 0.5 to about 10, 12, 15, or 20 IU/mL, or alternatively about 1 to 5 IU/mL, or about 2.5 to 25 IU/ml. In one aspect the IL-21 is present at a concentration of about 40 IU/mL, about 35 IU/mL, about 30 IU/mL, about 25 IU/mL, about 20 IU/mL, about 15 IU/mL, about 12 IU/mL, about 10 IU/mL, about 5 IU/mL, about 4 IU/mL, about 3 IU/mL, about 2 IU/mL, about 1 IU/mL or about 0.5 IU/mL. In one aspect the IL-21 is present at a concentration of about 0.5 IU/mL to about 50 IU/mL, preferably about 32.5 IU/mL.

The concentration of IL-2, IL-15 and/or IL-21 as referred to herein may be the initial concentration at the start of each expansion step. The IL-2, IL-15 and/or IL-21 concentration may remain constant throughout the culture step, for example by controlling the concentration with repeated feeding steps or may vary throughout the culture without exceeding the maximum concentration specified.

### Serum Replacement

Cells in in vitro culture are commonly supplemented with serum, for example human- or bovine-derived serum, in order to assist cell growth and maintenance. However, for GMP purposes in the production of therapeutic products intended for human administration, it is desirable not to include human- or bovine-derived serum if avoidable.

Alternatives to human or bovine-derived sera are commercially available in the form of serum replacement, for example CTS™ Immune Cell SR (Gibco).

A further option for serum replacement is the use of platelet lysate. Platelet lysate is a substitute supplement for fetal bovine serum (FBS) in cell culture. It is obtained from blood platelets after freeze/thaw cycles that cause the platelets to lyse, releasing growth factors supportive of cell expansion. FBS-free cell culture media containing platelet lysate are commercially available in GMP-quality and may be used in the manufacture of cell therapies. In a preferred aspect the platelet lysate is obtained from human blood, referred to herein as human platelet lysate (hPL).

Platelet lysate may be included in the cell culture medium at any of the T cell expansion steps defined herein. In one aspect, platelet lysate is present during the pre-expansion step. In another aspect, platelet lysate is present during the antigen-specific expansion step. In a yet further aspect, the platelet lysate is present during the non-specific boost expansion step. Preferably the platelet lysate is present throughout each of the steps.

In one aspect, platelet lysate is present at a concentration of about 1% to about 10%, for example about 5%.

### Antibodies

According to the method of the invention, the T cells may be cultured with antibodies as described herein.

The term “CD3” refers to cluster of differentiation 3. CD3 is a protein complex and T cell co-receptor that is involved in T cell activation. It is composed of a CD3γ chain, a CD30δ chain, and two CD3ε chains. These chains associate with the T cell receptor and the ζ-chain (zeta-chain) to generate an activation signal in T lymphocytes.

Binding of an anti-CD3 antibody to CD3 stimulates T-cell activation. Anti-CD3 antibodies are known in the art. For example, suitable anti-CD3 antibodies include, OKT3 (Muromab), TRX4 (Otelixizumab), PRV-031 (Teplizumab) and Visilizumab.

In one aspect, the anti-CD3 antibody is OKT3.

In one aspect, the anti-CD3 antibody is present at a concentration of about 0.1 to 1,000 ng/ml, e.g. about 10 to 1,000 ng/mL, for example about 30 to 300 ng/ml. In some embodiments, the cell culture medium comprises about 30 ng/ml of anti-CD3 antibody. In an embodiment, the cell culture medium comprises about 0.1 ng/ml, about 0.5 ng/ml, about 1 ng/ml, about 2.5 ng/ml, about 5 ng/ml, about 7.5 ng/ml, about 10 ng/ml. about 15 ng/ml, about 20 ng/ml, about 25 ng/ml, about 30 ng/ml, about 35 ng/ml, about 40 ng/mL, about 50 ng/ml, about 60 ng/ml, about 70 ng/ml, about 80 ng/ml, about 90 ng/ml, about 100 ng/mL, about 200 ng/ml, about 500 ng/ml, or about 1 μg/mL of anti-CD3 antibody. In an embodiment, the cell culture medium comprises between 0.1 ng/ml and 1 ng/ml, between 1 ng/ml and 5 ng/ml, between 5 ng/ml and ng/ml, between 10 ng/ml and 20 ng/ml, between 20 ng/ml and 30 ng/ml, between 30 ng/ml and 40 ng/ml, between 40 ng/ml and 50 ng/ml. or between 50 ng/mL and 100 ng/ml of anti-CD3 antibody.

The term “CD28” refers to Cluster of Differentiation 28. CD28 is constitutively expressed on naive T cells. Stimulation of CD28, for example by anti-CD28 antibodies, provides co-stimulatory signals required for T cell activation and survival. Suitable anti-CD28 antibodies are known in the art.

In one aspect, the anti-CD28 antibody is present at a concentration of about 0.1 to 1,000 ng/ml, e.g. about 10 to 1,000 ng/ml, for example about 30 to 300 ng/ml. In some embodiments, the cell culture medium comprises about 30 ng/ml of anti-CD28 antibody. In an embodiment, the cell culture medium comprises about 0.1 ng/ml, about 0.5 ng/mL, about 1 ng/ml, about 2.5 ng/ml, about 5 ng/mL, about 7.5 ng/ml, about 10 ng/ml. about 15 ng/mL, about 20 ng/ml, about 25 ng/ml, about 30 ng/ml, about 35 ng/mL, about 40 ng/mL, about 50 ng/ml, about 60 ng/ml, about 70 ng/mL, about 80 ng/ml, about 90 ng/ml, about 100 ng/ml, about 200 ng/ml, about 500 ng/ml, or about 1 μg/mL of anti-CD28 antibody. In an embodiment, the cell culture medium comprises between 0.1 ng/ml and 1 ng/ml, between 1 ng/ml and 5 ng/ml, between 5 ng/mL and ng/ml, between 10 ng/ml and 20 ng/ml, between 20 ng/mL and 30 ng/ml, between ng/ml and 40 ng/ml, between 40 ng/ml and 50 ng/ml. or between 50 ng/ml and 100 ng/ml of anti-CD28 antibody.

The term “CD2” refers to Cluster of Differentiation 2. CD2 is a cell adhesion molecule found on the surface of T cells and natural killer (NK) cells. In addition to its adhesive properties, CD2 also acts as a co-stimulatory molecule on T cells and NK cells. Suitable anti-CD2 antibodies are known in the art.

In one aspect, the anti-CD2 antibody is present at a concentration of about 0.1 to 1,000 ng/ml, e.g. about 10 to 1,000 ng/ml, for example about 30 to 300 ng/ml. In some embodiments, the cell culture medium comprises about 30 ng/ml of anti-CD2 antibody. In an embodiment, the cell culture medium comprises about 0.1 ng/ml, about 0.5 ng/mL, about 1 ng/ml, about 2.5 ng/ml, about 5 ng/ml, about 7.5 ng/ml, about 10 ng/ml. about 15 ng/ml, about 20 ng/ml, about 25 ng/ml, about 30 ng/ml, about 35 ng/ml, about 40 ng/ml, about 50 ng/ml, about 60 ng/ml, about 70 ng/ml, about 80 ng/ml, about 90 ng/ml, about 100 ng/mL, about 200 ng/mL, about 500 ng/mL, or about 1 μg/mL of anti-CD2 antibody. In an embodiment, the cell culture medium comprises between 0.1 ng/ml and 1 ng/ml, between 1 ng/mL and 5 ng/ml, between 5 ng/ml and ng/ml, between 10 ng/ml and 20 ng/ml, between 20 ng/ml and 30 ng/ml, between ng/mL and 40 ng/ml, between 40 ng/ml and 50 ng/ml. or between 50 ng/ml and 100 ng/ml of anti-CD2 antibody.

In one aspect, the non-specific boost expansion step uses anti-CD3 antibodies. In another aspect the non-specific boost expansion step uses a combination of anti-CD3 and anti-CD28 antibodies. In another aspect, the non-specific boost expansion step uses a combination of anti-CD3, anti-CD28 and anti-CD2 antibodies.

The anti-CD3 and/or anti-CD28 and/or anti-CD2 antibodies may be soluble, present on accessory cells, bound to a solid surface, for example, beads, or present in a polymeric nanomatrix structure or microspheres.

In a particular aspect of the invention, the antibodies are provided as soluble tetrameric antibody complexes. Binding of the tetrameric antibody complexes results in the cross-linking of cell surface ligands, thereby providing the required primary and co-stimulatory signals for T cell activation. Such antibody complexes are designed to activate and expand human T cells in the absence of magnetic beads, feeder cells or antigen.

In one aspect, a CD3/CD28 tetrameric antibody complex is used in any of the non-specific expansion steps described herein. Such complex is commercially available (e.g. ImmunoCult™ Human CD3/CD28 T cell Activator from STEMCELL Technologies, Inc.).

In one aspect, a CD3/CD28/CD2 tetrameric antibody complex is used in a non-specific expansion step. Such complex is commercially available (e.g. ImmunoCult™ Human CD3/CD28/CD2 T cell Activator from STEMCELL Technologies, Inc.).

In another aspect, the antibodies are conjugated to a colloidal polymeric nanomatrix which allows sterile filtration and excess reagent removal. A colloidal polymeric nanomatrix conjugated to humanised CD3 and CD28 antibodies is commercially available (e.g. T Cell TransAct™ human from Miltenyi Biotec).

In a further aspect, the antibodies are provided in microspheres, for example magnetic-free CD3/CD28 microspheres (e.g. Cloudz™ CD3/28 from Bio-Techne).

In a yet further aspect, magnetic beads are coated with the antibodies, for example anti-CD3 and anti-CD28 antibodies (e.g. Dynabeads™ Human T-Activator CD3/CD28 from Thermo Fisher Scientific).

In one aspect the invention provides a method for producing a population of T cells which comprises antigen-specific T cells, wherein said method comprises the steps of:
a) culturing isolated T cells in the presence of IL-2, IL-15 and IL-21; and
b) co-culturing said T cells with antigen presenting cells that have been loaded with antigen, wherein said T cells and antigen presenting cells are co-cultured in the presence of IL-2, and IL-15.

In another aspect the invention provides a method for producing a population of T cells which comprises antigen-specific T cells, wherein said method comprises the steps of:
a) culturing isolated T cells in the presence of IL-2 and IL-21;
b) co-culturing said T cells with antigen presenting cells that have been loaded with antigen, wherein said T cells and antigen presenting cells are co-cultured in the presence of IL-2; and
c) culturing the cells produced in step b) in the presence of anti-CD3 antibody, anti-CD28 antibody, anti-CD2 antibody and IL-2.

In another aspect the invention provides a method for producing a population of T cells which comprises antigen-specific T cells, wherein said method comprises the steps of:
a) culturing isolated T cells in the presence of IL-2, IL-15 and IL-21;
b) co-culturing said T cells with antigen presenting cells that have been loaded with antigen, wherein said T cells and antigen presenting cells are co-cultured in the presence of IL-2 and IL-15; and
c) culturing the cells produced in step b) in the presence of anti-CD3 antibody, anti-CD28 antibody, anti-CD2 antibody and IL-2.

In one aspect, the pre-expansion step a) lasts for about 7 to about 21 days, for example about 10 to about 18 days. In one aspect, the pre-expansion step lasts for about 11, 12, 13, 14, 15, 16 or 17 days.

In one aspect, the pre-expansion step a) includes additional components to increase the non-specific expansion of T cells. Addition of further components (as detailed below) to the pre-expansion step may result in an increased number of total T cells in the population, and preferably an increased number of antigen-specific T cells.

In one aspect, the pre-expansion step a) comprises culturing isolated T cells in the presence of one or more of:
(i) anti-CD3 antibody;
(ii) anti-CD28 antibody; and/or
(iii) anti-CD2 antibody.

In one aspect, the pre-expansion step uses anti-CD3 antibodies. In another aspect the pre-expansion step uses a combination of anti-CD3 and anti-CD28 antibodies. In another aspect, the pre-expansion step uses a combination of anti-CD3, anti-CD28 and anti-CD2 antibodies.

In one aspect, the pre-expansion step uses interferon gamma (IFNγ). Interferon gamma is a dimerized soluble cytokine that is the only member of the type II class of interferons and plays an important role in inducing and modulating an array of immune responses. Suitable types of IFNγ are known in the art and are commercially available, for example Human IFNγ Recombinant Protein from ThermoFisher and Recombinant Human IFN-γ from PeproTech.

In one aspect, the pre-expansion step uses anti-CD3 antibodies in combination with IFNγ. In another aspect the pre-expansion step uses a combination of anti-CD3 antibodies, anti-CD28 antibodies and IFNγ. In another aspect, the pre-expansion step uses a combination of anti-CD3, anti-CD28 and anti-CD2 antibodies and IFNγ.

In one aspect, the IFNγ is present at a concentration of about 0.1 to 1,000 ng/ml, e.g. about 10 to 500 ng/ml, for example about 5 to 20 ng/ml. In some embodiments, the cell culture medium comprises about 10 ng/ml of IFNγ. In an embodiment, the cell culture medium comprises about 0.1 ng/mL, about 0.5 ng/ml, about 1 ng/ml, about 2.5 ng/mL, about 5 ng/ml, about 7.5 ng/ml, about 10 ng/ml. about 15 ng/ml, about ng/ml, about 25 ng/ml, about 30 ng/ml, about 35 ng/mL, about 40 ng/ml, about 50 ng/ml, about 60 ng/ml, about 70 ng/ml, about 80 ng/mL, about 90 ng/mL, about 100 ng/ml, about 200 ng/ml, about 500 ng/ml, or about 1 μg/mL of IFNγ. In an embodiment, the cell culture medium comprises between 0.1 ng/ml and 1 ng/ml, between 1 ng/ml and 5 ng/mL, between 5 ng/ml and 10 ng/ml, between 10 ng/ml and 20 ng/ml, between 20 ng/ml and 30 ng/ml, between 30 ng/ml and 40 ng/mL, between 40 ng/ml and 50 ng/ml. or between 50 ng/ml and 100 ng/ml of IFNγ antibody.

The anti-CD3 antibodies and/or anti-CD28 antibodies and/or anti-CD2 antibodies and/or IFNγ may be added at any time point during the pre-expansion step. In one aspect, the additional components (antibodies and/or IFNγ) are added towards the end of the pre-expansion step, for example once 50%, 75% or more of the step has been completed. Accordingly, the antibodies and/or IFNγ may be added to the culture at day 5, 6, 7, 8, 9, 10, 11, 12, 13, 14 or 15 of the pre-expansion step.

In one aspect, the antigen-specific expansion step b) lasts for about 7 to about 21 days, for example about 10 to about 17 days. In one aspect, the specific expansion step lasts for about 8, 9, 10, 11, 12, 13, 14, 15, 16, 17 or 18 days.

In one aspect, the non-specific boost expansion step c) lasts for about 3 to about 21 days. In one aspect, the boost expansion step lasts for about 5, 6, 7, 8, 9, 10, 11, 12, 13, 14, 15, 16 or 17 days.

The cells may be split every 2-3 days to maintain appropriate cell density. Fresh cytokines may be added to maintain cytokine concentration.

### T Cell Population

The present invention further provides a T cell population produced by the methods of the invention.

T cell populations produced in accordance with the present invention may be enriched with T cells that are specific to, i.e. target, a given antigen. That is, the T cell population that is produced in accordance with the present invention will have an increased number of T cells that target one or more given antigens. For example, the T cell population of the invention will have an increased number of T cells that target said antigen compared with the T cells in the sample isolated from the subject. That is to say, the composition of the T cell population will differ from that of a “native” T cell population (i.e. a population that has not undergone the expansion steps discussed herein), in that the percentage or proportion of T cells that target said antigen will be increased.

The T cell population according to the invention may have at least about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100% T cells that target a given antigen or set of antigens. For example, the T cell population may have about 0.2%-5%, 5%-10%, 10-20%, 20-30%, 30-40%, 40-50%, 50-70% or 70-100% T cells that target a given antigen or set of antigens. In one aspect, the T cell population has at least about 1, 2, 3, 4 or 5% T cells that target said antigen(s), for example at least 20) about 2% or at least 2% T cells that target said antigen(s).

Alternatively put, the T cell population may have not more than about 5, 10, 15, 20, 25, 30, 35, 40, 45, 50, 55, 60, 65, 70, 75, 80, 85, 90, 91, 92, 93, 94, 95, 96, 97, 98, 99, 99.1, 99.2, 99.3, 99.4, 99.5, 99.6, 99.7, 99.8% T cells that do not target a given antigen. For example, the T cell population may have not more than about 95%-20 99.8%, 90%-95%, 80-90%, 70-80%, 60-70%, 50-60%, 30-50% or 0-30% T cells that do not target said antigen. In one aspect, the T cell population has not more than about 99, 98, 97, 96 or 95% T cells that do not target said antigen, for example not more than about 98% or 95% T cells that do not target said antigen.

An expanded population of antigen-reactive T cells may have a higher activity than a population of T cells not expanded, for example, using an antigen. Reference to “activity” may represent the response of the T cell population to restimulation with an antigenic peptide, e.g. a peptide corresponding to the peptide used for expansion, or a mix of antigen-derived peptides. Suitable methods for assaying the response are known in the art. For example, cytokine production may be measured (e.g. IL-2 or IFNγ production may be measured). The reference to a “higher activity” includes, for example, a 1-5, 5-10, 10-20, 20-50, 50-100, 100-500, 500-1000-fold increase in activity. In one aspect, the activity may be more than 1000-fold higher.

In a preferred embodiment, the invention provides a plurality or population, i.e. more than one, of T cells wherein the plurality of T cells comprises a T cell which recognises a given antigen and a T cell which recognises a different antigen. As such, the invention provides a plurality of T cells which recognise different antigens. Different T cells in the plurality or population may alternatively have different TCRs which recognise the same antigen.

In a preferred embodiment, the number of antigens recognised by the plurality of T cells is from 2 to 1000. For example, the number of antigens recognised may be 2, 3, 4, 5, 6, 7, 8, 9, 10, 20, 50, 100, 150, 200, 250, 300, 350, 400, 450, 500, 550, 600, 650, 700, 750, 800, 850, 900, 950 or 1000, preferably 2 to 100. There may be a plurality of T cells with different TCRs but which recognise the same antigen.

The T cell population may be all or primarily composed of CD8+ T cells, or all or primarily composed of a mixture of CD8+ T cells and CD4+ T cells or all or primarily composed of CD4+ T cells.

Helper T helper cells (TH cells) assist other white blood cells in immunologic processes, including maturation of B cells into plasma cells and memory B cells, and activation of cytotoxic T cells and macrophages. TH cells express CD4 on their surface (i.e. they are CD4+ T cells). TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytotoxic T cells (TC cells, or CTLs) destroy virally infected cells and tumour cells, and are also implicated in transplant rejection. CTLs express the CD8 at their surface (i.e. they are CD8+ T cells). These cells recognize their targets by binding to antigen associated with MHC class I, which is present on the surface of all nucleated cells. Through IL-10, adenosine and other molecules secreted by regulatory T cells, the CD8+ cells can be inactivated, which prevents autoimmune diseases.

### Functional Characteristics

In one aspect, the T cell population produced according to the methods of the invention has increased CD25 expression. The T cells may upregulate or increase expression of CD25 in response to restimulation with antigen.

The term “CD25” refers to the Interleukin-2 receptor alpha chain (IL2RA). The interleukin 2 receptor alpha and beta (IL2RB) chains, together with the common gamma chain (IL2RG), constitute the high-affinity IL2 receptor. Homodimeric alpha chains (IL2RA) result in low-affinity receptor, while homodimeric beta (IL2RB) chains produce a medium-affinity receptor. CD25 is expressed with CD4 on regulatory T cells.

In one aspect, the T cell population produced according to the methods of the invention has increased CD27 expression. CD27 is a member of the tumour necrosis factor receptor superfamily. CD27 binds CD70, resulting in differentiation and clonal expansion of T cells. CD27 plays a role in the generation of T cell memory.

In one aspect, the T cell population produced according to the methods of the invention has decreased CD57 expression. The CD57 antigen is present on subsets of peripheral blood mononuclear cells, NK lymphocytes and T lymphocytes. CD57 expression on human lymphocytes may indicate an inability to proliferate (senescence), though CD57 positive cells may also display high cytotoxic potential, memory-like features and potent effector functions.

As discussed herein, T cells produced according to the invention may have increased expression of IFNγ. Suitable methods for determining expression of IFNγ are known in the art.

T cells as described herein may have the CD3+/CD56− phenotype.

In another aspect, the T cell population produced according to the methods of the invention may have a more even balance or ratio of CD4+ and CD8+ T cells. For example, the methods of the invention as described herein may result in a population of T cells which contains a higher proportion of CD8+ cells than previous methods. An increase in CD8+ cells may be advantageous (for example, see Prieto et al, J Immunother 2010 June; 33(5):547-56). The T cell population may therefore be more balanced for CD4+/CD8+ T cells than a T cell population achieved by previous methods. In one aspect, the T cell population may contain from about 20% to about 80% CD8+ T cells, such as from about 30% to 70% CD8+ T cells, for example at least about 10%, 15%, 20%, 25%, 30%, 35%, 40%, 45%, 50%, 55%, 60%, 65%, 70%, 75%, 80%, 85%, 90% or 95% or more CD8+ T cells. In one embodiment the T cell population comprises at least about 50% CD8+ T cells.

### T Cell Composition

The present invention further provides a T cell composition which comprises a population of T cells according to the invention as described herein.

The T cell composition may be a pharmaceutical composition comprising a plurality of T cells as defined herein. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### Antigen-Specific T Cells

Identification of antigen-specific T cells in a mixed starting population of T cells may be performed using methods which are known in the art. For example, antigen-specific T cells may be identified using MHC multimers comprising an antigenic peptide.

MHC multimers are oligomeric forms of MHC molecules, designed to identify and isolate T-cells with high affinity to specific antigens amid a large group of unrelated T-cells. Multimers may be used to display class 1 MHC, class 2 MHC, or nonclassical molecules (e.g. CD1d).

The most commonly used MHC multimers are tetramers. These are typically produced by biotinylating soluble MHC monomers, which are typically produced recombinantly in eukaryotic or bacterial cells. These monomers then bind to a backbone, such as streptavidin or avidin, creating a tetravalent structure. These backbones are conjugated with fluorochromes to subsequently isolate bound T-cells via flow cytometry, for example.

### Antigens

In one aspect of the invention the T cell population comprises T cells which target cancer-associated or tumour-specific antigens.

Tumour antigens include the following: CEA, immature laminin receptor, TAG-72, HPV E6 and E7, BING-4, calcium-activated chloride channel 2, cyclin-B1, 9D7, Ep-CAM, EphA3, Her2/neu, telomerase, mesothelin, SAP-1, survivin, BAGE family, CAGE family, GAGE family, MAGE family, SAGE family, XAGE family, NY-ESO-1/LAGE-1, PRAME, SSX-2, Melan-A/MART-1, gp100/pmel17, tyrosinase, TRP-1/-2, P.polypeptide, MC1R, prostate-specific antigen, beta-catenin, BRCA1/2, CDK4, CML66, fibronectin, MART-2, p53, ras, TGF-betaRII and MUC1.

Tumour antigens may also include the following: 707-AP=707 alanine proline, AFP=alpha (α)-fetoprotein, ART-4=adenocarcinoma antigen recognized by T cells 4, BAGE=B antigen; β-catenin/m, β-catenin/mutated, Bcr-abl=breakpoint clusterregion-Abelson, CAMEL=CTL-recognized antigen on melanoma, CAP-1=carcinoembryonic antigen peptide-1, CASP-8=caspase-8, CDC27m=cell-division-cycle 27 mutated, CDK4/m=cycline-dependent kinase 4 mutated, CEA=carcinoembryonic antigen, CT=cancer/testis (antigen), Cyp-B=cyclophilin B, DAM=differentiation antigen melanoma (the epitopes of DAM-6 and DAM-10 are equivalent, but the gene sequences are different. DAM-6 is also called MAGE-B2 and DAM-10 is also called MAGE-B1), ELF2M=elongation factor 2 mutated, ETV6-AML1=Etsvariant gene 6/acute myeloid leukemia 1 gene ETS, G250=glycoprotein 250, GAGE=G antigen, GnT-V=N-acetylglucosaminyltransferase V, Gp100=glycoprotein 100 kD, HAGE=helicose antigen, HER-2/neu=human epidermal receptor-2/neurological, HLA-A*0201-R170I=arginine (R) to isoleucine (I) exchange at residue 170 of the α-helix of the α2-domain in the HLA-A2 gene, HPV-E7=human papilloma virus E7, HSP70-2M=heat shock protein 70-2 mutated, HST-2=human signet ring tumor-2, hTERT or hTRT=human telomerase reverse transcriptase, iCE=intestinal carboxylesterase, KIAA0205=name of the gene as it appears in databases, LAGE=L antigen, LDLR/FUT=low density lipid receptor/GDP-L-fucose: β-D-galactosidase 2-α-L-fucosyltransferase, MAGE=melanoma antigen, MART-1/Melan-A=melanomaantigen recognized by T cells-1/Melanoma antigen A, MC1R=melanocortin 1 receptor, Myosin/m=myosin mutated üMUC1=mucin 1, MUM-1, −2, −3=melanomaubiquitous mutated 1, 2, 3, NA88-A=NA cDNA clone of patient M88, NY-ESO-1=New York-esophageous 1, P15=protein 15, p190 minor bcr-abl=protein of 190 3KD bcr-abl, Pml/RARα=promyelocytic leukaemia/retinoic acid receptor α, PRAME=preferentially expressed antigen of melanoma, PSA=prostate-specific antigen, PSM=prostate-specific membrane antigen, RAGE=renal antigen, RU1 or RU2=renalubiquitous 1 or 2, SAGE=sarcoma antigen, SART-1 or SART-3=squamous antigenrejecting tumor 1 or 3, TEL/AML1=translocation Ets-family leukemia/acute myeloidleukemia 1, TPI/m=triosephosphate isomerase mutated, TRP-1=tyrosinase relatedprotein 1, or gp75, TRP-2=tyrosinase related protein 2, TRP-2/INT2=TRP-2/intron2, WT1=Wilms' tumor gene.

### Neoantigens

In one aspect of the invention the antigen may be a neoantigen.

A “neoantigen” is a tumour-specific antigen which arises as a consequence of a mutation within a cancer cell. Thus, a neoantigen is not expressed (or expressed at a significantly lower level) by healthy (i.e. non-tumour) cells in a subject. A neoantigen may be processed to generate distinct peptides which can be recognised by T cells when presented in the context of MHC molecules. As described herein, neoantigens may be used as the basis for cancer immunotherapies. References herein to “neoantigens” are intended to include also peptides derived from neoantigens. The term “neoantigen” as used herein is intended to encompass any part of a neoantigen that is immunogenic.

An “antigen” as referred to herein is a molecule which itself, or a part thereof, is capable of stimulating an immune response, when presented to the immune system or immune cells in an appropriate manner. The binding of a neoantigen to a particular MHC molecule (encoded by a particular HLA allele) may be predicted using methods which are known in the art. Examples of methods for predicting MHC binding include those described by Lundegaard et al., O'Donnel et al., and Bullik-Sullivan et al. For example, MHC binding of neoantigens may be predicted using the netMHC (Lundegaard et al.) and netMHCpan (Jurtz et al.) algorithms. Binding of a neoantigen to a particular MHC molecule is a prerequisite for the neoantigen to be presented by said MHC molecule on the cell surface.

The neoantigen described herein may be caused by any non-silent mutation (whether coding or non-coding) which alters a protein and/or its expression in a cancer cell compared to the non-mutated protein expressed by a wild-type, healthy cell. In other words, the mutation results in the expression of an amino acid sequence that is not expressed, or expressed at a very low level in a wild-type, healthy cell. For example, the mutation may occur in the coding sequence of a protein, thus altering the amino acid sequence of the resulting protein. This may be referred to as a “coding mutation”. As another example, the mutation may occur in a splice site, thus resulting in the production of a protein that contains a set of exons that is different or less common in the wild-type protein. As a further example, the mutated protein may result from a translocation or fusion.

A “mutation” refers to a difference in a nucleotide sequence (e.g. DNA or RNA) in a tumour cell compared to a healthy cell from the same individual. The difference in the nucleotide sequence can result in the expression of a protein which is not expressed by a healthy cell from the same individual. In embodiments, the mutation may be one or more of a single nucleotide variant (SNV), a multiple nucleotide variant (MNV), a deletion mutation, an insertion mutation, an indel mutation, a frameshift mutation, a translocation, a missense mutation, a splice site mutation, a fusion, or any other change in the genetic material of a tumour cell.

An “indel mutation” refers to an insertion and/or deletion of bases in a nucleotide sequence (e.g. DNA or RNA) of an organism. Typically, the indel mutation occurs in the DNA, preferably the genomic DNA, of an organism. In embodiments, the indel may be from 1 to 100 bases, for example 1 to 90, 1 to 50, 1 to 23 or 1 to 10 bases. An indel mutation may be a frameshift indel mutation. A frameshift indel mutation is an insertion or deletion of one or more nucleotides that causes a change in the reading frame of the nucleotide sequence. Such frameshift indel mutations may generate a novel open-reading frame which is typically highly distinct from the polypeptide encoded by the non-mutated DNA/RNA in a corresponding healthy cell in the subject.

The mutations may be identified by exome sequencing, RNA-seq, whole genome sequencing and/or targeted gene panel sequencing and/or routine Sanger sequencing of single genes. Suitable methods are known in the art. Descriptions of exome sequencing and RNA-seq are provided by Boa et al. (Cancer Informatics. 2014; 13(Suppl 2):67-82.) and Ares et al. (Cold Spring Harb Protoc. 2014 Nov. 3; 2014(11):1139-48); respectively. Descriptions of targeted gene panel sequencing can be found in, for example, Kammermeier et al. (J Med Genet. 2014 November; 51(11):748-55) and Yap K L et al. (Clin Cancer Res. 2014. 20:6605). See also Meyerson et al., Nat. Rev. Genetics, 2010 and Mardis, Annu Rev Anal Chem, 2013. Targeted gene sequencing panels are also commercially available (e.g. as summarised by Biocompare ((http://www.biocompare.com/Editorial-Articles/161194-Build-Your-Own-Gene-Panels-with-These-Custom-NGS-Targeting-Tools/)).

Sequence alignment to identify nucleotide differences (e.g. SNVs) in DNA and/or RNA from a tumour sample compared to DNA and/or RNA from a non-tumour sample may be performed using methods which are known in the art. For example, nucleotide differences compared to a reference sample may be performed using the method described by Koboldt et al. (Genome Res. 2012; 22: 568-576). The reference sample may be the germline DNA and/or RNA sequence.

### Clonal Neoantigens

In one aspect the neoantigen may be a clonal neoantigen.

A “clonal neoantigen” (also sometimes referred to as a “truncal neoantigen”) is a neoantigen arising from a clonal mutation. A “clonal mutation” (sometimes referred to as a “truncal mutation”) is a mutation that is present in essentially every tumour cell in one or more samples from a subject (or that can be assumed to be present in essentially every tumour cell from which the tumour genetic material in the sample(s) is derived). Thus, a clonal mutation may be a mutation that is present in every tumour cell in one or more samples from a subject. For example, a clonal mutation may be a mutation which occurs early in tumorigenesis.

A “subclonal neoantigen” (also sometimes referred to as a “branched neoantigen”) is a neoantigen arising from a subclonal mutation. A “subclonal mutation” (also sometimes referred to as a “branch mutation”) is a mutation that is present in a subset or a proportion of cells in one or more tumour samples from a subject (or that can be assumed to be present in a subset of the tumour cells from which the tumour genetic material in the sample(s) is derived). For example, a subclonal mutation may be the result of a mutation occurring in a particular tumour cell later in tumorigenesis, which is found only in cells descended from that cell.

The wording “essentially every tumour cell” in relation to one or more samples of a subject may refer to at least 70%, at least 75%, at least 80%, at least 85%, at least 90%, at least 91%, at least 92%, at least 93%, at least 94% at least 95%, at least 96%, at least 97%, at least 98%, or at least 99% of the tumour cells in the one or more samples or the subject.

As such, a clonal neoantigen is a neoantigen which is expressed effectively throughout a tumour. A subclonal neoantigen is a neoantigen that is expressed in a subset or a proportion of cells or regions in a tumour. ‘Expressed effectively throughout a tumour’ may mean that the clonal neoantigen is expressed in all regions of the tumour from which samples are analysed.

It will be appreciated that a determination that a mutation is ‘encoded (or expressed) within essentially every tumour cell’ refers to a statistical calculation and is therefore subject to statistical analysis and thresholds.

Likewise, a determination that a clonal neoantigen is ‘expressed effectively throughout a tumour’ refers to a statistical calculation and is therefore subject to statistical analysis and thresholds.

Various methods for determining whether a neoantigen is “clonal” are known in the art. Any suitable method may be used to identify a clonal neoantigen, for example as described in Landau et al. (Cell. 2013 Feb. 14; 152(4):714-26); MacGranahan et al. (Science 2016 Mar. 25; 351(6280): 1463-1469); or Roth et al. (Nat Methods. 2014 April; 11(4): 396-398).

By way of example, the cancer cell fraction (CCF), describing the proportion of cancer cells that harbour a mutation, may be used to determine whether mutations are clonal or subclonal. For example, the cancer cell fraction may be determined by integrating variant allele frequencies with copy numbers and purity estimates as described by Landau et al. (Cell. 2013 Feb. 14; 152(4):714-26).

Suitably, CCF values may be calculated for all mutations identified within each and every tumour region analysed. If only one region is used (i.e. only a single sample), only one set of CCF values will be obtained. This will provide information as to which mutations are present in all tumour cells within that tumour region and will thereby provide an indication if the mutation is clonal or subclonal. If multiple tumour regions are used (e.g. multiple samples), a CCF value may be obtained individually for each region or jointly for one or more of the multiple tumour regions.

Such a CCF estimate can also be used to identify mutations that are likely to be clonal. A clonal mutation may be defined as a mutation which has a cancer cell fraction (CCF) ≥0.75, such as a CCF ≥0.80, 0.85. 0.90, 0.95 or 1.0. A subclonal mutation may be defined as a mutation which has a CCF <0.95, 0.90, 0.85, 0.80, or 0.75. In one aspect, a clonal mutation is defined as a mutation which has a CCF ≥0.95 and a subclonal mutation is defined as a mutation which has a CCF <0.95.

As stated, determining a clonal mutation is subject to statistical analysis and threshold. A CCF estimate may be associated with (e.g. derived from) a distribution associating a probability density with each of a plurality of possible values of CCF between 0 and 1, from which statistical estimates of confidence may be obtained. For example, a mutation may be defined as likely to be a clonal mutation if the 95% CCF confidence interval is >=0.75, i.e. the upper bound of the 95% confidence interval of the estimated CCF is greater than or equal to 0.75. In other words, a mutation may be defined as likely to be a clonal mutation if there is an interval of CCF with lower bound L and upper bound H that is such that P(L<CCF<H)=95% with H>=0.75.

In one aspect a mutation may be defined as a clonal mutation if the 95% confidence interval of the CCF includes CCF=1.

In another aspect a mutation may be identified as clonal if there is more than a 50% chance or probability that its cancer cell fraction (CCF) reaches or exceeds the required value as defined above, for example 0.75 or 0.95, such as a chance or probability of 55%, 60%, 65%, 70%, 75%, 80%, 85%, 90%, 95% or more. In other words, a mutation may be identified as clonal if P(CCF>0.75)>=0.5.

Probability values may be expressed as percentages or fractions. The probability may be defined as a posterior probability.

In one aspect, a mutation may be identified as clonal if the probability that the mutation has a cancer cell fraction greater than 0.95 is ≥0.75.

In another aspect, a mutation may be identified as clonal if there is more than a 50% chance that its cancer cell fraction (CCF) is ≥0.95.

In a further aspect, mutations may be classified as clonal or subclonal based on whether the posterior probability that their CCF exceeds a first threshold (e.g. 0.95) is greater or lesser than a second threshold (e.g. 0.5), or that their CCF=1 is greater or lesser than a third threshold, respectively.

In another aspect a mutation may be identified as clonal if the probability that the mutation has a cancer cell fraction greater than 0.75 is ≥0.5.

In one aspect the T cell population may comprise T cells which target a plurality i.e. more than one clonal neo-antigen.

In one aspect the number of clonal neoantigens is 2-1000. For example, the number of clonal neoantigens may be 2, 3, 4, 5, 6, 7, 8, 9, 10, 20, 50, 100, 150, 200, 250, 300, 350, 400, 450, 500, 550, 600, 650, 700, 750, 800, 850, 900, 950 or 1000, for example the number of clonal neoantigens may be from 2 to 100.

In one aspect, the T cell population comprises a T cell which recognises a clonal neoantigen and a T cell which recognises a different clonal neoantigen. As such, the T cell population may comprise a plurality of T cells which recognise different clonal neoantigens.

In one aspect the number of clonal neoantigens recognised by the population of T cells is 2-1000. For example, the number of clonal neoantigens recognised may be 2, 3, 4, 5, 6, 7, 8, 9, 10, 20, 50, 100, 150, 200, 250, 300, 350, 400, 450, 500, 550, 600, 650, 700, 750, 800, 850, 900, 950 or 1000, for example the number of clonal neoantigens recognised may be from 2 to 100.

In one aspect the T cells recognise the same clonal neoantigen.

In one aspect the neoantigen may be a subclonal neoantigen as described herein.

As described above, a clonal neoantigen is one which is encoded within essentially every tumour cell, that is the mutation encoding the neoantigen is present within essentially every tumour cell and is likely to be expressed effectively throughout the tumour. However, a clonal neoantigen may be predicted to be presented by an HLA molecule encoded by an HLA allele which is lost in at least part of a tumour. In this case, the clonal neoantigen may not actually be presented on essentially every tumour cell. As such, the presentation of the neoantigen may not be clonal, i.e. it is not presented within essentially every tumour cell. Methods for predicting loss of HLA are described in International Patent Publication No. WO2019/012296.

In one aspect of the invention as described herein the neoantigen is predicted to be presented within essentially every tumour cell (i.e. the presentation of the neoantigen is clonal).

### Neoantigen-Specific T Cell Therapy

The T cell population according to the invention may comprise T cells which target neoantigens. In one aspect of the invention, the T cell population may comprise T cells which target clonal neoantigens. In the context of the present invention, the term “target” may mean that the T cell is specific for, and mounts a response to, the neoantigen.

In one aspect the T cell population may comprise T cells which have been selectively expanded to target neoantigens, such as clonal neoantigens.

That is, the T cell population may have an increased number of T cells that target one or more neoantigens. For example, the T cell population of the invention will have an increased number of T cells that target a neoantigen compared with the T cells in the sample isolated from the subject. That is to say, the composition of the T cell population will differ from that of a “native” T cell population (i.e. a population that has not undergone the identification and expansion steps discussed herein), in that the percentage or proportion of T cells that target a neoantigen will be increased, and/or the ratio of T cells in the population that target neoantigens to T cells that do not target neoantigens will be higher in favour of the T cells that target neoantigens.

The T cell population according to the invention may have at least about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100% T cells that target a neoantigen. For example, the T cell population may have about 0.2%-5%, 5%-10%, 10-20%, 20-30%, 30-40%, 40-50%, 50-70% or 70-100% T cells that target a neoantigen. In one aspect the T cell population has at least about 1, 2, 3, 4 or 5% T cells that target a neoantigen, for example at least about 2% or at least 2% T cells that target a neoantigen.

Alternatively put, the T cell population may have not more than about 5, 10, 15, 20, 25, 30, 35, 40, 45, 50, 55, 60, 65, 70, 75, 80, 85, 90, 91, 92, 93, 94, 95, 96, 97, 98, 99, 99.1, 99.2, 99.3, 99.4, 99.5, 99.6, 99.7, 99.8% T cells that do not target a neoantigen. For example, the T cell population may have not more than about 95%-99.8%, 90%-95%, 80-90%, 70-80%, 60-70%, 50-60%, 30-50% or 0-30% T cells that do not target a neoantigen. In one aspect the T cell population has not more than about 99, 98, 97, 96 or 95% T cells that do not target a neoantigen, for example not more than about 98% or 95% T cells that do not target a neoantigen.

An expanded population of neoantigen-reactive T cells may have a higher activity than a population of T cells not expanded, for example, using a neoantigen peptide. Reference to “activity” may represent the response of the T cell population to restimulation with a neoantigen peptide, e.g. a peptide that comprises part or all of the peptide (or corresponding coding sequence) used for expansion, or a mix of neoantigen peptides. Suitable methods for assaying the response are known in the art. For example, cytokine production may be measured (e.g. IL-2 or IFNγ production may be measured). The reference to a “higher activity” includes, for example, a 1-5, 5-10, 10-20, 20-50, 50-100, 100-500, 500-1000-fold increase in activity. In one aspect the activity may be more than 1000-fold higher.

In one aspect of the invention, T cells that are capable of specifically recognising one or more neoantigens are identified in a sample from the subject and then expanded by ex vivo culture as described herein. Identification of neoantigen-specific T cells in a mixed starting population of T cells may be performed using methods which are known in the art. For example, neoantigen-specific T cells may be identified using MHC multimers comprising a neoantigen peptide as described herein.

MHC multimers are oligomeric forms of MHC molecules, designed to identify and isolate T-cells with high affinity to specific antigens amid a large group of unrelated T-cells. Multimers may be used to display class 1 MHC, class 2 MHC, or nonclassical molecules (e.g. CD1d).

The most commonly used MHC multimers are tetramers. These are typically produced by biotinylating soluble MHC monomers, which are typically produced recombinantly in eukaryotic or bacterial cells. These monomers then bind to a backbone, such as streptavidin or avidin, creating a tetravalent structure. These backbones are conjugated with fluorochromes to subsequently isolate bound T-cells via flow cytometry, for example.

### Immunotherapy

The invention as described herein may provide a T cell population for use in therapy, particularly immunotherapy.

The invention encompasses a T cell population or T cell therapy as described herein for use in the prevention or treatment of cancer in a subject.

The invention encompasses a method for treating a subject with cancer wherein said method comprises administering to said subject a T cell population or T cell therapy as described herein.

The invention also encompasses a T cell population or T cell therapy as described herein for use in the manufacture of a medicament for use in the prevention or treatment of cancer in a subject.

The invention further encompasses use of a T cell population or T cell therapy as described herein in the prevention or treatment of cancer in a subject.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy can include adoptive T cell therapy, autologous T cell therapy, tumour-infiltrating lymphocyte (TIL) therapy, engineered T cell therapy, chimeric antigen receptor (CAR) T cell therapy, engineered TCR T cell therapy and allogeneic T cell transplantation. Examples of T cell therapies are described in International Publication Nos, WO2018/002358, WO2013/088114, WO2015/077607, WO2015/143328, WO2017/049166 and WO2011/140170.

The T cells of the immunotherapy may originate from any source known in the art. For example, T cells may be differentiated in vitro from a hematopoietic stem cell population, or T cells can be obtained from a subject. T cells may be obtained from, e.g., peripheral blood mononuclear cells, bone marrow, lymph node tissue, cord blood, thymus tissue, tissue from a site of infection, ascites, pleural effusion, spleen tissue, and tumours. In addition, the T cells may be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by reference in its entirety.

The invention as described herein also encompasses use of the T cell population according to the invention in the treatment or prevention of cancer in a subject.

The T cell population as described herein may be referred to as a T cell therapy.

A single dose of T cell therapy may be administered to the patient. In one aspect a single dose of T cell therapy is administered to the patient on day 0 only. In other aspects of the invention, multiple doses of T cell therapy are administered to the patient starting from day 0. For example, the number of doses of T cell therapy may be 2, 3, 4, 5, 6, 7, 8, 9, 10 or more than 10 doses.

Dosing may be once, twice, three times, four times, five times, six times, or more than six times per year. Alternatively, dosing may be once, twice, three times, four times, five times, six times, or more than six times per month. In a further aspect dosing may be once, twice, three times, four times, five times, six times, or more than six times every two weeks. In yet a further aspect dosing may be once, twice, three times, four times, five times, six times, or more than six times per week, for example once a week, or once every other day.

Administration of the T cell therapy may continue as long as necessary.

The T cell therapy as described herein may be used in vitro, ex vivo or in vivo, for example either for in situ treatment or for ex vivo treatment followed by the administration of the treated cells to the body.

In certain aspects according to the invention as described herein the T cell therapy is reinfused into a subject, for example following T cell isolation and expansion as described herein. Suitable methods for reinfusing T cells are known in the art.

The T cell therapy may be administered to a subject at a suitable dose. The dosage regimen may be determined by the attending physician and clinical factors. It is accepted in the art that dosages for any one patient depend upon many factors, including the patient's size, body surface area, age, the particular compound to be administered, sex, time and route of administration, general health, and other drugs being administered concurrently.

The T cell therapy may involve the transfer of a given number of T cells as described herein to a patient. The therapeutically effective amount of T cells may be at least about 10³ cells, at least about 10⁴ cells, at least about 10⁵ cells, at least about 10⁶ cells, at least about 10⁷ cells, at least about 10⁸ cells, at least about 10⁹ cells, at least about 10¹⁰ cells, at least about 10¹¹ cells, at least about 10¹² or at least about 10¹³ cells. 20)

Other suitable doses of T cells may be as described in, for example, WO 2016/191755, WO2019/112932, WO2018/226714, WO2018/182817, WO2018/129332, WO2018/129336, WO2018/094167, WO2018/081789 and WO2018/081473.

### Modified T Cells

In one aspect of the invention the T cells may be modified T cells, for example genetically modified T cells.

A method for expanding T cells according to the present invention may further comprise a step of modifying, e.g. by gene-editing, at least a portion of the T cells.

The T cells may be modified by gene-editing methods. Gene editing methods are known in the art, and may be selected from a CRISPR method, a TALE method, a zinc finger method, and a combination thereof.

In one aspect gene-editing may cause expression of one or more immune checkpoint genes to be silenced or reduced, e.g. selected from the group comprising PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD 160, TIGIT, CD96, CRT AM, LAIR1, SIGLEC7, SIGLEC9, CD244, TNFRSF10B, TNFRSF10A, CASP8, C ASP 10, CASP3, CASP6, CASP7, FADD, FAS, SMAD2, SMAD3, SMAD4, SMAD10, SKI, SKIL, TGIFI, IL10RA, IL10RB, HMOX2, IL6R, IL6ST, EIF2AK4, CSK, PAG1, SIT1, FOXP3, PRDMI, BATF, GUCY1A2, GUCY1A3, GUCY1B2, GUCY1B3, TOX, ANKRD11, SOCS1, and BCOR.

In another aspect, the gene-editing may cause expression of one or more immune checkpoint genes to be enhanced, e.g. selected from the group comprising CCR2, CCR4, CCR5, CXCR2, CXCR3, CX3CR1, IL-2, IL-4, IL-7, IL-10, IL-15, IL-21, the NOTCH 1/2 intracellular domain (ICD), and/or the NOTCH ligand mDLLI.

Methods for gene-editing are described in WO2021/081378.

### Cancer

In one aspect the cancer as described herein is selected from lung cancer (small cell, non-small cell and mesothelioma), melanoma, bladder cancer, gastric cancer, oesophageal cancer, breast cancer (e.g. triple negative breast cancer), colorectal cancer, cervical cancer, ovarian cancer, endometrial cancer, kidney cancer (renal cell), brain cancer (e.g. gliomas, astrocytomas, glioblastomas), lymphoma, small bowel cancers (duodenal and jejunal), leukaemia, liver cancer (hepatocellular carcinoma), pancreatic cancer, hepatobiliary tumours, germ cell cancers, prostate cancer, merkel cell carcinoma, head and neck cancers (squamous cell), thyroid cancer, high microsatellite instability (MSI-H), and sarcomas.

In one aspect the cancer is selected from melanoma and non small cell lung cancer (NSCLC).

In one aspect the cancer, such as melanoma or NSCLC, may be metastatic, and/or inoperable and/or recurrent.

Treatment according to the present invention may also encompass targeting circulating tumour cells and/or metastases derived from the tumour.

### Subject

The terms “subject” and “patient” are used interchangeably herein.

In a preferred aspect of the present invention, the subject is a mammal, preferably a cat, dog, horse, donkey, sheep, pig, goat, cow, mouse, rat, rabbit or guinea pig, but most preferably the subject is a human.

As defined herein “treatment” refers to reducing, alleviating or eliminating one or more symptoms of the disease which is being treated, relative to the symptoms prior to treatment.

“Prevention” (or prophylaxis) refers to delaying or preventing the onset of the symptoms of the disease. Prevention may be absolute (such that no disease occurs) or may be effective only in some individuals or for a limited amount of time.

### Dosing Regimen

In one aspect of the invention as described herein, a single dose of T cell therapy is administered to the patient. In one aspect a single dose of T cell therapy is administered to the patient on day 0 only. In other aspects of the invention, multiple doses of T cell therapy are administered to the patient starting from day 0. For example, the number of doses of T cell therapy may be 2, 3, 4, 5, 6, 7, 8, 9, 10 or more than 10 doses.

Dosing may be once, twice, three times, four times, five times, six times, or more than six times per year. Alternatively, dosing may be once, twice, three times, four times, five times, six times, or more than six times per month. In a further aspect dosing may be once, twice, three times, four times, five times, six times, or more than six times every two weeks. In yet a further aspect dosing may be once, twice, three times, four times, five times, six times, or more than six times per week, for example once a week, or once every other day.

Administration of the T cell therapy may continue as long as necessary.

### IL-2 Therapy

A T cell population or therapy according to the present invention as described herein may be used in combination with IL-2 administration, for example in the treatment of cancer in a patient.

In one aspect the invention provides a T cell therapy according to the present invention and a dose of IL-2 of less than about 2.0 MIU/m²/day for use in the treatment or prevention of cancer in a patient. In a further aspect the invention provides a T cell therapy for use in the treatment or prevention of cancer in a patient, wherein said T cell therapy is for administration with IL-2, and wherein said IL-2 is for administration at a dose of less than about 2.0 MIU/m²/day.

The T cell therapy and IL-2 may be for separate, simultaneous or sequential administration to the patient.

The IL-2 may be administered at a dose of about 1.9 MIU/m²/day, about 1.8 MIU/m²/day, about 1.7 MIU/m²/day, about 1.6 MIU/m²/day, about 1.5 MIU/m²/day, about 1.4 MIU/m²/day, about 1.3 MIU/m²/day, about 1.2 MIU/m²/day, about 1.1 MIU/m²/day, about 1.0 MIU/m²/day, about 0.9 MIU/m²/day, about 0.8 MIU/m²/day, about 0.7 MIU/m²/day, about 0.6 MIU/m²/day, about 0.5 MIU/m²/day, about 0.4 MIU/m²/day, about 0.3 MIU/m²/day or about 0.2 MIU/m²/day.

In one aspect said IL-2 is administered at a dose of about 1.0 MIU/m²/day.

In a further aspect said IL-2 is administered once daily.

In another aspect said IL-2 is administered daily for about 14, 13, 12, 11, 10, 9, 8, 7, 6, 5, 4, 3, 2, or 1 days, preferably 10 days.

In one aspect said IL-2 is administered for less than 14 days, for example about 13, 12, 11, 10, 9, 8, 7, 6, 5, 4, 3, 2, or 1 days, preferably 10 days. In one aspect said IL-2 is administered for not more than 13 days, for example not more than 12, 11, 10, 9, 8, 7, 6, 5, 4, 3, 2, or 1 day.

Said dose of IL-2 may be the same each day.

In one aspect of the invention the total dose of IL-2 administered to said patient does not exceed about 10 MIU/m².

In one aspect the first dose of said IL-2 is administered on the same day as the T cell therapy.

In one aspect, less than 14 doses of said IL-2 are administered to said patient. For example, 13, 12, 11, 10, 9, 8, 7, 6, 5, 4, 3, 2, or 1 doses of said IL-2 are administered to said patient.

In a preferred aspect, 10 doses of said IL-2 are administered to said patient.

In a further aspect said IL-2 is administered daily on days 0 to 9.

The IL-2 can be administered by any route, including intravenously (IV) and subcutaneously (SC). Low-dose IL-2 is typically given by subcutaneous injection, whereas high-dose IL-2 is generally administered via i.v. infusion. In one particular aspect, the IL-2 is administered subcutaneously.

### Lymphodepletion

Prior to transfer of T cells, patients typically undergo a lymphodepletion therapy. Lymphodepletion treatment improves the efficacy of T cell therapy by reducing the number of endogenous lymphocytes and increasing the serum level of homeostatic cytokines and/or pro-immune factors present in the patient. Examples of non-myeloablative lymphodepletion regimens for immunotherapy are disclosed in International Patent Publication No. WO 2004/021995.

In one aspect, the present invention includes administration of a lymphodepleting agent, such as cyclophosphamide and/or fludarabine. In one aspect the invention includes the administration of cyclophosphamide and fludarabine prior to a T cell therapy. The timing of the administration of each component can be adjusted to maximize effect. As described herein, the day that a T cell therapy is administered may be designated as day 0. The cyclophosphamide and fludarabine may be administered at any time prior to administration of the T cell therapy.

In one aspect, the administration of the cyclophosphamide and fludarabine begins at least seven days, at least six days, at least five days, at least four days, at least three days, at least two days, or at least one day prior to the administration of the T cell therapy.

In another aspect, the administration of the cyclophosphamide and fludarabine may begin at least eight, nine, ten, eleven, twelve, thirteen or fourteen days prior to the administration of the T cell therapy. In one aspect, the administration of the cyclophosphamide and fludarabine begins seven, six, or five days prior to the administration of the T cell therapy. In one particular aspect, administration of the cyclophosphamide begins about seven days prior to the administration of the T cell therapy, and the administration of the fludarabine begins about five days prior to the administration of the T cell therapy. In another aspect, administration of the cyclophosphamide begins about five days prior to the administration of the T cell therapy, and the administration of the fludarabine begins about five days prior to the administration of the T cell therapy.

The timing of the administration of each component can be adjusted to maximize effect. In general, the cyclophosphamide and fludarabine can be administered daily for about two, three, four, five, six or seven days. As described herein, the day the T cell therapy is administered to the patient may be designated as day 0. In some aspects, the cyclophosphamide is administered to the patient on day 7 and day 6 prior to day 0 (i.e., day −7 and day −6). In other aspects, the cyclophosphamide is administered to the patient on day −5, day −4, and day −3. In some aspects, the fludarabine is administered to the patient on day −5, day −4, day −3, day −2, and day −1. In other aspects, the fludarabine is administered to the patient on day −5, day −4, and day −3. The cyclophosphamide and fludarabine can be administered on the same or different days. In one particular aspect, the cyclophosphamide and fludarabine are both administered to the patient on day −6, day −5 and day −4.

The cyclophosphamide and fludarabine can be administered by any route, including intravenously (IV). In some aspects, the cyclophosphamide is administered by IV over about 30 to 120 minutes.

In one particular aspect, the invention includes a method of conditioning a patient in need of a T cell therapy comprising administering to the patient a dose of cyclophosphamide of about 500 mg/m²/day and a dose of fludarabine of about 60 mg/m²/day, wherein the cyclophosphamide is administered on days −5, −4, and −3, and wherein the fludarabine is administered on days −5, −4, and −3.

In another aspect, the invention includes a method of conditioning a patient in need of a T cell therapy comprising administering to the patient a dose of cyclophosphamide of about 300 or 500 mg/m²/day and a dose of fludarabine of about 30 or 60 mg/m²/day, wherein the cyclophosphamide is administered on days −7 and −6, and wherein the fludarabine is administered on days −5, −4, −3, −2, and −1.

In one aspect the lymphodepleting agent is administered daily for 3 days. In one aspect the lymphodepleting agent is administered on days −6, −5 and −4 prior to administration of said T cell therapy. In one aspect cyclophosphamide is administered at a dose of between about 200 mg/m2/day and about 500 mg/m2/day, preferably at a dose of about 300 mg/m2/day. In one aspect fludarabine is administered at a dose of between about 20 mg/m2/day and 50 mg/m2/day, preferably at a dose of about 30 mg/m2/day.

In one aspect fludarabine is administered at a dose of about 30 mg/m2 and cyclophosphamide is administered at a dose of about 300 mg/m2 on each of days −6, −5, and −4 prior to cell infusion.

In one aspect the invention provides a method of treating cancer in a patient, comprising administering to the patient:
(i) a lymphodepleting regimen of about 300 mg/m2/day of cyclophosphamide and about 30 mg/m2/day of fludarabine prior to administration of said T cell therapy;
(ii) a single dose of T cell therapy; and
(iii) a dose of IL-2 of about 1.0 MIU/m²/day administered once daily for about 10 days wherein the first dose of said IL-2 is administered on the same day as the T cell therapy.

### Other Combination Therapies

The invention as described herein may also be combined with other suitable therapies.

The methods and uses for treating cancer according to the present invention may be performed in combination with additional cancer therapies. In particular, the T cell compositions according to the present invention may be administered in combination with checkpoint blockade therapy, co-stimulatory antibodies, chemotherapy and/or radiotherapy, targeted therapy or monoclonal antibody therapy.

Checkpoint inhibitors include, but are not limited to, PD-1 inhibitors, PD-L1 inhibitors, Lag-3 inhibitors, Tim-3 inhibitors, TIGIT inhibitors, BTLA inhibitors and CTLA-4 inhibitors, for example. Co-stimulatory antibodies deliver positive signals through immune-regulatory receptors including but not limited to ICOS, CD137, CD27 OX-40 and GITR. In a preferred embodiment the checkpoint inhibitor is a CTLA-4 inhibitor.

Examples of suitable immune checkpoint inhibitors include pembrolizumab, nivolumab, atezolizumab, durvalumab, avelumab, tremelimumab and ipilimumab.

A chemotherapeutic entity as used herein refers to an entity which is destructive to a cell, that is the entity reduces the viability of the cell. The chemotherapeutic entity may be a cytotoxic drug. A chemotherapeutic agent contemplated includes, without limitation, alkylating agents, anthracyclines, epothilones, nitrosoureas, ethylenimines/methylmelamine, alkyl sulfonates, alkylating agents, antimetabolites, pyrimidine analogs, epipodophylotoxins, enzymes such as L-asparaginase; biological response modifiers such as IFNα, IL-2, G-CSF and GM-CSF; platinum coordination complexes such as cisplatin, oxaliplatin and carboplatin, anthracenediones, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen as such diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; non-steroidal antiandrogens such as flutamide; and drug-conjugates with a chemotherapeutic agent payload.

‘In combination’ may refer to administration of the additional therapy before, at the same time as or after administration of the T cell composition according to the present invention.

In one aspect, the T cell compositions according to the present invention may be administered in combination with a checkpoint blockade therapy. The checkpoint inhibitor may be administered both before and after administration of the T cell composition. In a particular embodiment, one dose of the checkpoint inhibitor is administered before the T cell composition, and another dose is administered 2 weeks after the T cell composition and further doses continue for up to 12 months. In a preferred embodiment, the checkpoint inhibitor is pembrolizumab.

In addition or as an alternative to the combination with checkpoint blockade, the T cell composition of the present invention may also be genetically modified to render them resistant to immune-checkpoints using gene-editing technologies including but not limited to TALEN and Crispr/Cas. Such methods are known in the art, see e.g. US 20140120622. Gene editing technologies may be used to prevent the expression of immune checkpoints expressed by T cells including but not limited to PD-1, Lag-3, Tim-3, TIGIT, BTLA CTLA-4 and combinations of these. The T cell as discussed here may be modified by any of these methods.

The T cell according to the present invention may also be genetically modified to express molecules increasing homing into tumours and or to deliver inflammatory mediators into the tumour microenvironment, including but not limited to cytokines, soluble immune-regulatory receptors and/or ligands.

### Kit

In one aspect the invention provides a kit comprising a T cell therapy as described herein.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this disclosure.

This disclosure is not limited by the exemplary methods and materials disclosed herein, and any methods and materials similar or equivalent to those described herein can be used in the practice or testing of aspects of this disclosure. Numeric ranges are inclusive of the numbers defining the range.

The headings provided herein are not limitations of the various aspects or aspects of this disclosure which can be had by reference to the specification as a whole. Accordingly, the terms defined immediately below are more fully defined by reference to the specification as a whole.

The term “protein”, as used herein, includes proteins, polypeptides, and peptides.

Other definitions of terms may appear throughout the specification. Before the exemplary aspects are described in more detail, it is to understand that this disclosure is not limited to particular aspects described, as such may, of course, vary. It is also to be understood that the terminology used herein is for the purpose of describing particular aspects only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of as used herein are synonymous with “including”, “includes” or “containing”, “contains”, and are inclusive or open-ended and do not exclude additional, non-recited members, elements or method steps. The terms “comprising”, “comprises” and “comprised of also include the term “consisting of”.

The publications discussed herein are provided solely for their disclosure prior to the filing date of the present application. Nothing herein is to be construed as an admission that such publications constitute prior art to the claims appended hereto.

The invention will now be further described, by way of example only, with reference to the following Examples.

### Examples

### Example 1-Identification and Production of Antigen

Blood and tumour samples were obtained from each patient and whole exome sequencing (WES) was carried out. The proprietary PELEUS™ bioinformatics platform was used to carry out the following steps:
(i) identify patient-specific somatic mutations (including single nucleotide variants (SNVs), multiple nucleotide variants (MNVs) and insertions/deletions (indels)) by comparing DNA sequence data from the germline (blood) sample and the matched tumour samples to each other and to a reference genome;
(ii) identify a set of mutations that are likely to be clonal in view of the sequence data from the patient using a Bayesian approach (see e.g. McGranahan et al., Science Vol 135:6280, p. 1463-1469; Roth et al., Nat Methods. 2014 April; 11(4): 396-398); and
(iii) design a set of peptides comprising the set of somatic mutations identified as likely to be clonal.

The resulting set of candidate antigenic peptides was manufactured using standard peptide synthesis methods. Each peptide sequence was 29 amino acids long and comprised one of the clonal somatic mutations and part of the germline sequence that surrounds the somatic mutation. Between 70 and 185 clonal neoantigen peptides were produced per patient for use in the antigen-specific expansion step.

### Example 2-Production of Dendritic Cells

Blood samples were obtained from each patient and peripheral blood mononuclear cells (PBMC) were separated using density gradient centrifugation. Monocytes were enriched by positive selection of CD14+ cell using a human magnetic antibody cell sorting system (Miltenyi Biotec) according to the manufacturer's procedure. Monocytes were differentiated into immature dendritic cells using GM-CSF and IL-4 and then matured with TNFα, II-1β, IL-6 and PGE2. Finally, the dendritic cells were washed and loaded with patient-specific peptides.

### Example 3-Expansion of T Cells

TIL were expanded using the following protocols:

### Generation 1.2

Tumour fragments were cultured in vitro in TexMACS media containing IL-2 (6000 IU/mL) and IL-21 (32.5 IU/ml) for 14 days (pre-expansion). TIL were subsequently co-cultured with peptide-loaded dendritic cells in media containing IL-2 (100 IU/mL) for 17 days (antigen-specific expansion).

### Generation 1.6

Tumour fragments were cultured in vitro in TexMACS media containing IL-2 (6000 IU/mL) and IL-21 (32.5 IU/mL) for 14 days (pre-expansion). TIL were subsequently co-cultured with peptide-loaded dendritic cells in media containing IL-2 (100 IU/mL) for 10 days (antigen-specific expansion). TIL were then further expanded in media containing a 1/200 dilution of ImmunoCult™ Human CD3/CD28/CD2 T Cell Activator and IL-2 (4000 IU/mL) for 7 days (non-specific boost).

### Generation 2.0

Tumour fragments were cultured in vitro in TexMACS media containing IL-2 (6000 IU/mL), IL-15 (160 IU/mL), IL-21 (32.5 IU/mL) and platelet lysate for 14 days (pre-expansion). TIL were subsequently co-cultured with peptide-loaded dendritic cells in media containing IL-2 (100 IU/mL), IL-15 (160 IU/mL) and platelet lysate for 17 days (antigen-specific expansion).

### Generation 2.6

Tumour fragments were cultured in vitro in TexMACS media containing IL-2 (6000 IU/mL), IL-15 (160 IU/mL), IL-21 (32.5 IU/mL) and platelet lysate for 14 days (pre-expansion). TIL were subsequently co-cultured with peptide-loaded dendritic cells in media containing IL-2 (100 IU/mL), IL-15 (160 IU/mL) and platelet lysate for 10 days (antigen-specific expansion). TIL were then further expanded in media containing a 1/200 dilution of ImmunoCult™ Human CD3/CD28/CD2 T Cell Activator, platelet lysate, IL-15 (10 ng/ml) and IL-2 (4000 IU/mL) for 7 days (non-specific boost).

### Example 4-Functional Characterisation of Antigen-Specific T Cells

The total number of T cells (CD3+CD56−) at day 0 and day 17 of the co-culture was determined by flow cytometry using the 6-color TBNK Reagent with BD Trucount™ (BD Biosciences). T cell numbers were scaled based on the tumour weight used in each condition and the total weight of the tumour excision.

The percentage of clonal neoantigen reactive T cells (cNeT) present was measured by flow cytometry following restimulation with peptide pools and intracellular cytokine staining. Reactivity was defined as the percentage of IFNγ and/or TNFα expressing T cells (CD3+). ELISpot following restimulation with single peptides was used to determine the number of different clonal neoantigen reactivities present in the cell populations.

Cell phenotype was assessed by flow cytometry following staining for CD3, CD56, CD4, CD8, CD45RA, CD197, CD25, CD27 and CD57. Memory phenotype was defined by CD45RA and CD197 expression (Naïve=CD45RA⁺CD197⁺, Central memory=CD45RA⁻CD197⁺, Effector memory=CD45RA⁻CD197⁻, TEMRA=CD45RA⁺CD197⁺). For some experiment's cells were restimulated with peptide pools prior to staining.

### Results

We have completed a side-by-side, matched pair analysis comparing three different dose-boosting strategies and determined that the Gen 2.6 process generates ˜10-fold higher doses of clonal neoantigen T cells (cNeT) compared to the Gen 1.2 process. Gen 2.6 generates a lower percentage (˜2-fold) of cNeT compared to Gen 1.2 but this is compensated by the significantly higher (>10-fold) total T cell number delivered by the process. Gen 2.6 is able to generate functionally fit cells producing equivalent amounts of the key functional marker IFNγ.

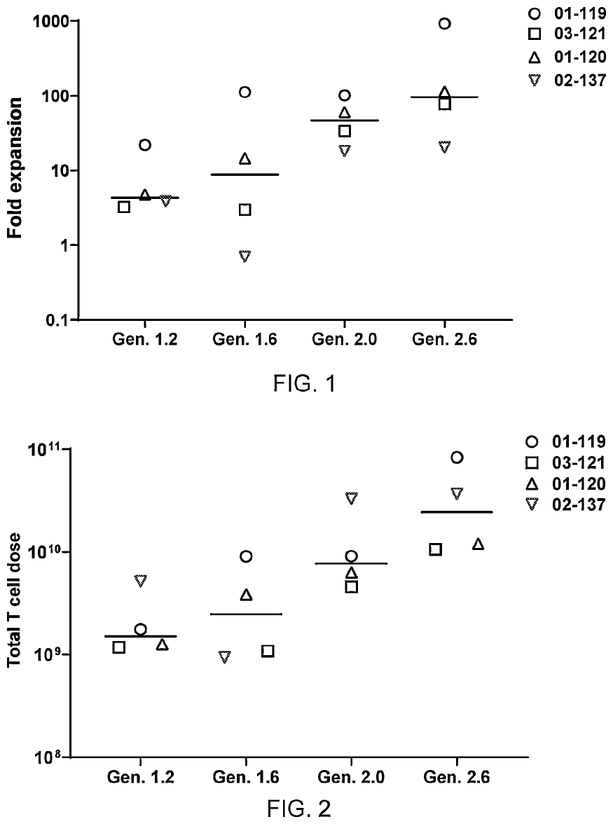

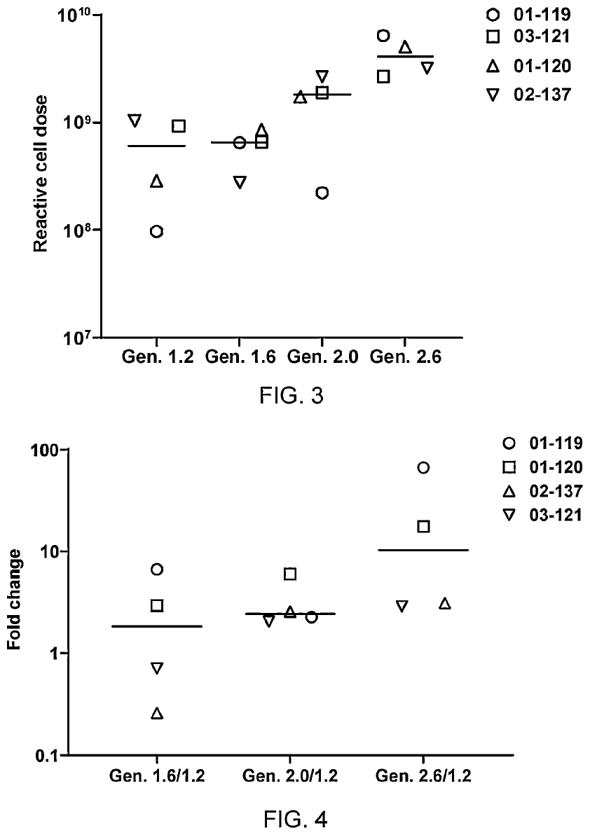

Gen 2.6 gives the greatest expansion of total T cells in the co-culture (FIG. 1) and the greatest T (CD3+CD56−) cell dose (FIG. 2). Gen 2.6 also generated the highest number of reactive cells, and therefore potential cNeT dose (FIG. 3). Gen 2.6 boosted over 15-fold the lowest Gen 1.2 cNeT doses. While two of the runs only gave a low fold change in cNeT dose, these were from the runs that worked very well for Gen1.2. The very large fold changes were in products with a very poor reactivity in Gen 1.2 so effectively Gen2.6 rescued these products (FIG. 4).

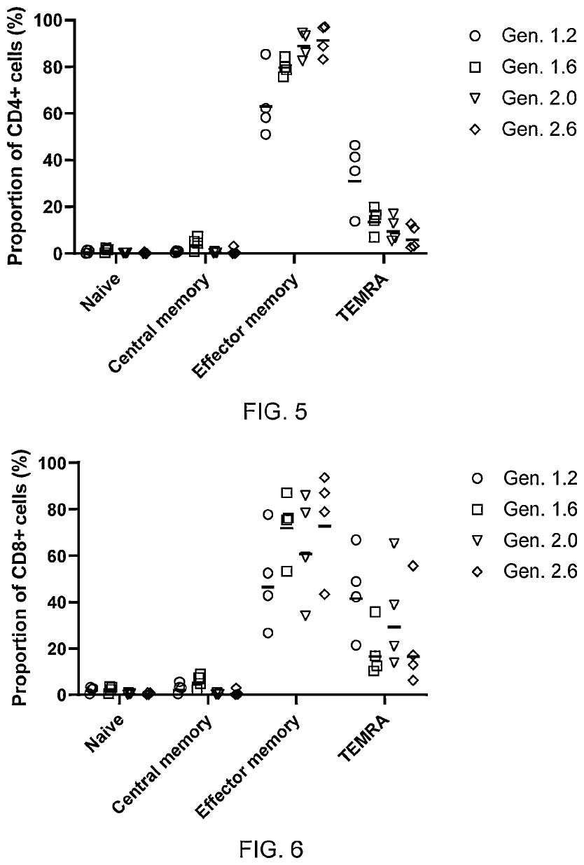

The Gen 2.6 process predominantly generates the desired effector memory T cell phenotype associated with the cytotoxic phenotype, in both CD8+ and CD4+ T cells (FIG. 5).

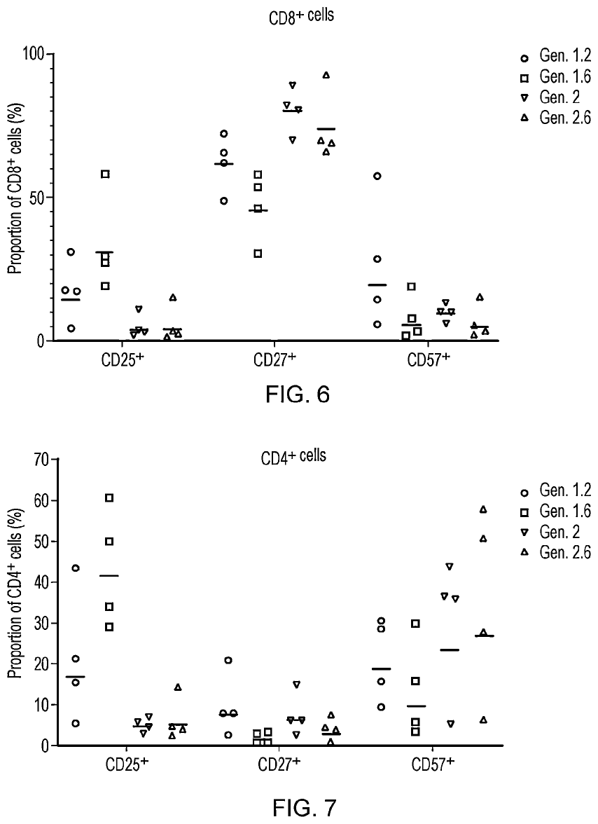

The Gen 2.6 process delivers highly fit T cells with minimal impact on phenotypic fitness relative to Gen 1.2. T cell products produced by the Gen 2.6 process showed higher expression of activation marker CD27, and lower expression of exhaustion marker CD57 in CD8⁺ T cells. However, Gen 2.6 also showed lower expression of CD25, the IL-2 receptor (FIG. 6).

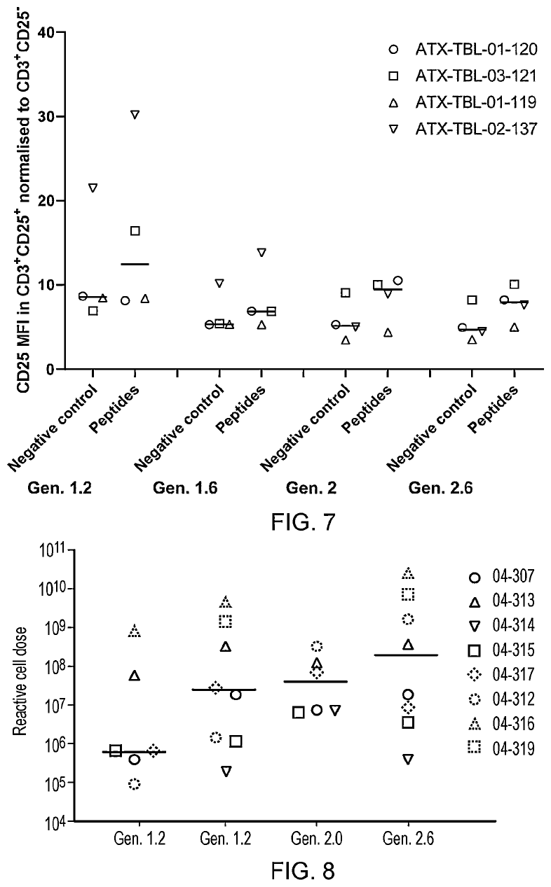

However, subsequent experiments showed that cells generated by the Gen 2.6 process are still capable of CD25 upregulation in response to peptide restimulation, suggesting sensitivity to IL-2 is retained (FIG. 7).

In conclusion, these results demonstrate the ability to increase total T cell dose using a non-specific boost expansion step after an antigen-specific expansion step, while also retaining T cell fitness and functionality. Reactivity to clonal neoantigen peptides is retained in the product leading to a boost in the dose of cNeT.

### Example 5-Expansion of T Cells

cNeT were generated from tumour samples obtained from cancer patients (n=8) using each process described above. As before, Gen 2.6 generated the highest reactive cell dose (FIG. 8).

### Example 6-Non-Specific Boost During Pre-Expansion Step

TIL were expanded using the following protocols:

### Generation 2.8.1

Tumour fragments were cultured in vitro in TexMACS media containing a 1/200 dilution of ImmunoCult™ Human CD3/CD28/CD2 T Cell Activator, IL-2 (6000 IU/mL), IL-15 (160 IU/mL), IL-21 (22.5 IU/mL) and platelet lysate for 14 days (pre-expansion). TIL were subsequently co-cultured with peptide-loaded dendritic cells in media containing IL-2 (100IU/mL), IL-15 (160 IU/mL) and platelet lysate for 10 days (antigen-specific expansion). TIL were then further expanded in media containing a 1/200 dilution of ImmunoCult™ Human CD3/CD28/CD2 T Cell Activator, platelet lysate, IL-15 (160 IU/ml) and IL-2 (3000-6000 IU/mL) for 7 days (non-specific boost).

### Generation 2.8.2

Tumour fragments were cultured in vitro in TexMACS media containing a 1/200 dilution of ImmunoCult™ Human CD3/CD28/CD2 T Cell Activator, IL-2 (6000 IU/mL), IL-15 (160 IU/mL), IL-21 (22.5 IU/mL), IFNγ (20 ng/ml) and platelet lysate for 14 days (pre-expansion). TIL were subsequently co-cultured with peptide-loaded dendritic cells in media containing IL-2 (100IU/mL), IL-15 (160 IU/mL) and platelet lysate for 10 days (antigen-specific expansion). TIL were then further expanded in media containing a 1/200 dilution of ImmunoCult™ Human CD3/CD28/CD2 T Cell Activator, platelet lysate, IL-15 (160 IU/ml) and IL-2 (3000-6000 IU/mL) for 7 days (non-specific boost).

### Results

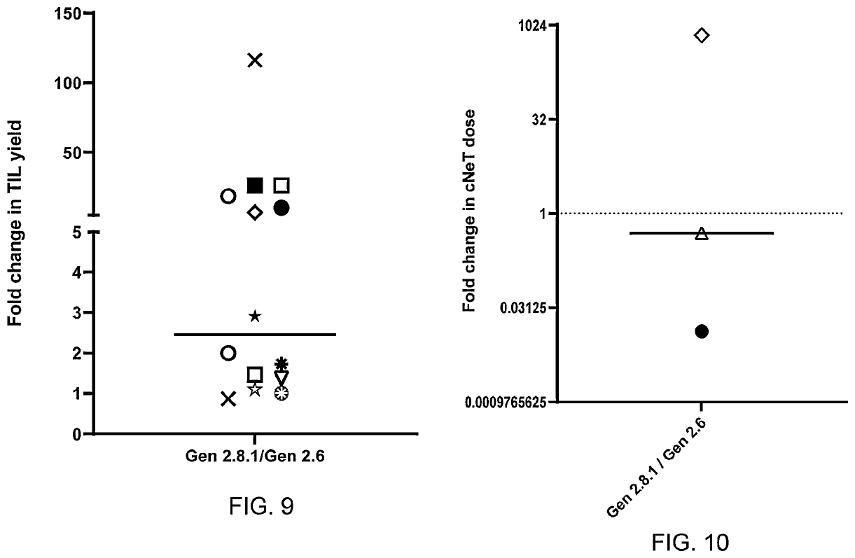

Addition of ImmunoCult™ Human CD3/CD28/CD2 T Cell Activator during the pre-expansion (Gen 2.8.1) generated a ˜2.5-fold higher number of TIL at the end of pre-expansion compared to Gen 2.6 (FIG. 9). The cNeT dose generated by Gen 2.6 and Gen 2.8.1 was similar, in ⅓ patients dose was greatly increased (˜700 fold) and in ⅔ patients it was reduced (FIG. 10).

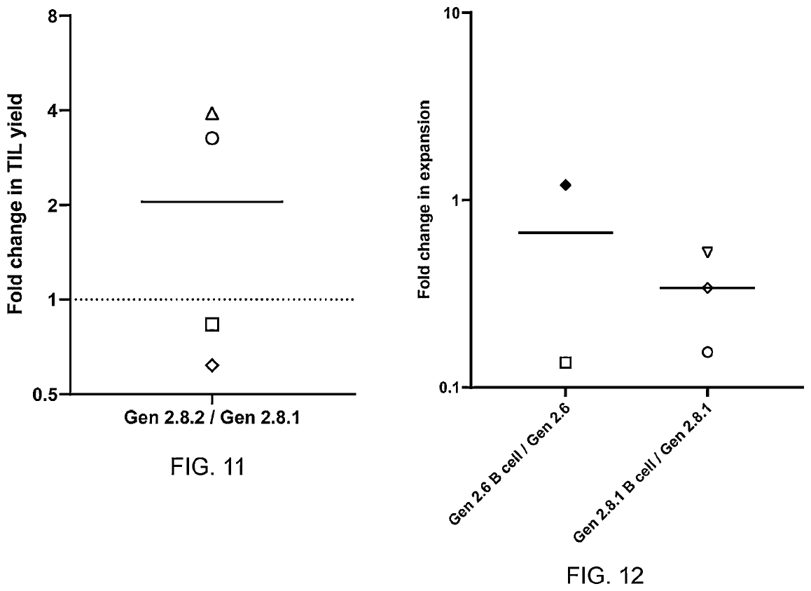

In 2/4 patients, addition of IFNγ in combination with ImmunoCult™ Human CD3/CD28/CD2 T Cell Activator (Gen 2.8.2) during the pre-expansion phase of the process increased TIL yield compared to addition of ImmunoCult™ Human CD3/CD28/CD2 T Cell Activator alone (FIG. 11).

### Example 7-Expansion of B Cells and Use as APCs

### Activation and Expansion of B Cells

Blood samples were obtained from each patient and peripheral blood mononuclear cells (PBMC) were separated using density gradient centrifugation. B cells were enriched by positive selection of CD19+ cells using a human magnetic antibody cell sorting system (Miltenyi Biotec) according to the manufacturer's procedure. B cells were activated and expanded for 14 days in culture with 12 IU/ml CD40L, 4.6 μg/ml CpG (MACS® GMP CpG-P, Miltenyi Biotec) and 50 ng/ml IL-4. Finally, the B cells were loaded with patient-specific peptides.

### Generation 2.6 B Cells

Tumour fragments were cultured in vitro in TexMACS media containing a 1/200 dilution of ImmunoCult™ Human CD3/CD28/CD2 T Cell Activator, IL-2 (6000 IU/mL), IL-15 (160 IU/mL), IL-21 (22.5 IU/mL) and platelet lysate for 14 days (pre-expansion). TIL were subsequently co-cultured with peptide-loaded, activated B cells in media containing IL-2 (100IU/mL), IL-15 (160 IU/mL) and platelet lysate for 10 days (antigen-specific expansion). TIL were then further expanded in media containing a 1/200 dilution of ImmunoCult™ Human CD3/CD28/CD2 T Cell Activator, platelet lysate, IL-(160 IU/ml) and IL-2 (3000-6000 IU/mL) for 7 days (non-specific boost).

### Generation 2.8.1 B Cells

Tumour fragments were cultured in vitro in TexMACS media containing a 1/200 dilution of ImmunoCult™ Human CD3/CD28/CD2 T Cell Activator, IL-2 (6000 IU/mL), IL-15 (160 IU/mL), IL-21 (22.5 IU/mL) and platelet lysate for 14 days (pre-expansion). TIL were subsequently co-cultured with peptide-loaded, activated B cells in media containing IL-2 (100IU/mL), IL-15 (160 IU/mL) and platelet lysate for 10 days (antigen-specific expansion). TIL were then further expanded in media containing a 1/200 dilution of ImmunoCult™ Human CD3/CD28/CD2 T Cell Activator, platelet lysate, IL-(160 IU/ml) and IL-2 (3000-6000 IU/mL) for 7 days (non-specific boost).

### Results

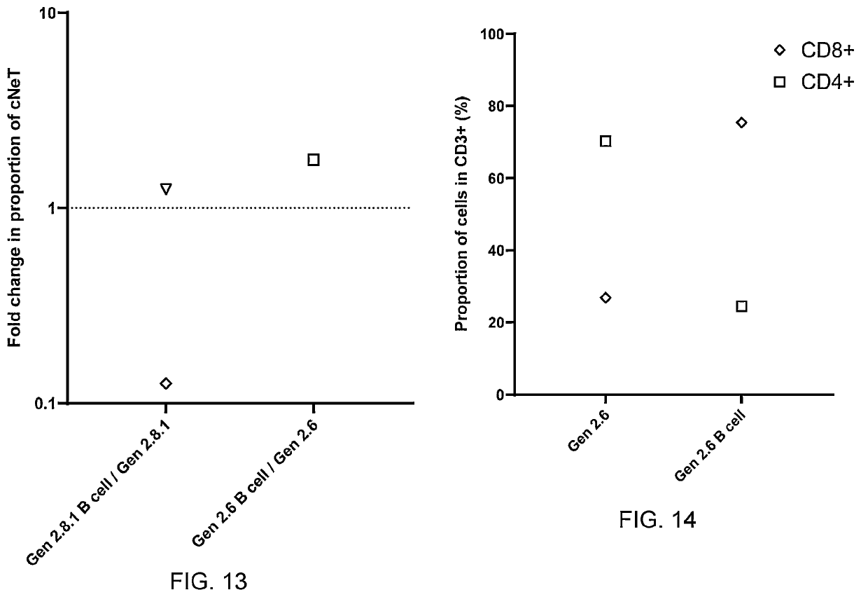

CD40 activated B cells can be used as an alternative to dendritic cells during the antigen specific expansion phase of the process. Co-culture with peptide-pulsed B cells (Gen 2.6 B cell and Gen 2.8.1 B cell) resulted in lower T cell expansion than co-culture with dendritic cells (Gen 2.6 and Gen 2.8.1) as shown in FIG. 12. Similar proportions of cNeT were present following expansion with B cells compared to expansion with DCs (FIG. 13). In a Gen 2.6 product where most cells were CD4+ the corresponding Gen 2.6 B cell product was mostly CD8+ (FIG. 14).

1. A method for producing a population of T cells which comprises antigen-specific T cells, wherein said method comprises an antigen-specific T cell expansion step followed by a non-specific T cell boost expansion step.

2. The method according to claim 1, wherein said method comprises:
a) an antigen-specific expansion step comprising co-culturing isolated T cells with antigen presenting cells that have been loaded with antigen, wherein said T cells and antigen presenting cells are co-cultured in the presence of IL-2; and
b) a non-specific boost expansion step comprising culturing the cells produced in step a) in the presence of anti-CD3 antibodies and/or anti-CD28 antibodies and/or IL-2.

3. The method according to claim 1 or 2, further comprising a non-specific pre-expansion step prior to the antigen-specific expansion step, preferably comprising culturing isolated T cells in the presence of IL-2 and IL-21.

4. The method according to claim 3, wherein the non-specific pre-expansion step further comprises culturing the T cells in the presence of anti-CD3 antibodies, anti-CD28 antibodies, anti-CD2 antibodies and/or IFNγ.

5. The method according to any preceding claim, wherein the non-specific pre-expansion and/or antigen-specific expansion steps further comprise culturing the T cells in the presence of IL-15.

6. The method according to any preceding claim wherein said method comprises the steps of:
a) an antigen-specific expansion step comprising co-culturing said T cells with antigen presenting cells that have been loaded with antigen, wherein said T cells and antigen presenting cells are co-cultured in the presence of IL-2 and IL-15; and
b) a non-specific boost expansion step comprising culturing the cells produced in step a) in the presence of anti-CD3 antibodies and/or anti-CD28 antibodies and/or IL-2.

7. The method according to any preceding claim, comprising the steps of:
a) a non-specific pre-expansion step comprising culturing isolated T cells in the presence of IL-2, IL-15 and IL-21;
b) an antigen-specific expansion step comprising co-culturing the T cells produced in step a) with antigen presenting cells that have been loaded with antigen, wherein said T cells and antigen presenting cells are co-cultured in the presence of IL-2 and IL-15; and
c) a non-specific boost expansion step comprising culturing the cells produced in step b) in the presence of anti-CD3 antibodies and/or anti-CD28 antibodies and/or IL-2.

8. The method according to any preceding claim, wherein the non-specific boost expansion step comprises culturing the T cells in the presence of anti-CD3 antibodies and IL-2, preferably in the presence of anti-CD3 antibodies, anti-CD28 antibodies and IL-2.

9. The method according to any preceding claim, wherein the non-specific boost expansion step comprises culturing the T cells in the presence of anti-CD3 antibodies, anti-CD28 antibodies, anti-CD2 antibodies and IL-2.

10. The method according to any preceding claim, wherein the non-specific pre-expansion step comprises culturing the T cells in the presence of IL-2, IL-15, IL-21, anti-CD3 antibodies, anti-CD28 antibodies and anti-CD2 antibodies.

11. The method according to claim 10, wherein the pre-expansion step further comprises culturing the T cells in the presence of IFNγ.

12. The method according to any preceding claim, wherein the non-specific pre-expansion and/or antigen-specific expansion and/or non-specific boost expansion steps further comprise culturing the T cells in the presence of platelet lysate.

13. The method according to any preceding claim, wherein the IL-21 in the non-specific pre-expansion step is present at a concentration of about 0.5 to 50 IU/mL, preferably about 32.5 IU/mL;
and/or wherein the IL-2 in the non-specific pre-expansion step is present at a concentration of about 1,000 to 10,000 IU/mL, preferably about 6,000 IU/mL;
and/or wherein the IL-2 in the antigen-specific expansion step is present at a concentration of about 10 to 500 IU/mL, preferably about 100 IU/mL;
and/or wherein the IL-2 in the non-specific boost expansion step is present at a concentration of about 1,000 to 10,000 IU/mL preferably about 4,000 IU/mL; and/or
wherein the IL-15 is present at a concentration of about 10 to 16,000 IU/mL, preferably about 160 IU/mL.

14. The method according to any preceding claim, wherein the culture period of the non-specific pre-expansion step is a period of about 7 to about 21 days, preferably about 10 to 18 days, more preferably about 14 to 16 days;
and/or the culture period of the antigen-specific expansion step is a period of about 7 to 21 days, preferably about 10 to 17 days;
and/or the culture period of the non-specific boost expansion step is a period of about 3 to about 21 days, preferably about 7 to 17 days.

15. The method according to any preceding claim, wherein the antigen-presenting cells have been loaded with a tumour antigen, and/or wherein the antigen-presenting cells are dendritic cells and/or B cells.

16. The method according to any preceding claim wherein the antigen is a neoantigen, preferably a clonal neoantigen.

17. A T cell population obtained or obtainable by the method according to any preceding claim or a T cell composition comprising said T cell population, wherein preferably said population or composition comprises at least about 10×10⁶ antigen-specific T cells or at least about 0.2%-5%, 5%-10%, 10-20%, 20-30%, 30-40%, 40-50%, 50-70% or 70-100% antigen-specific T cells.

18. A T cell population or composition according to claim 17 for use in treating or preventing cancer in a subject, wherein preferably said cancer is bladder cancer, gastric, oesophageal, breast cancer, colorectal cancer, cervical cancer, ovarian cancer, endometrial cancer, kidney cancer (renal cell), lung cancer (small cell, non-small cell and mesothelioma), brain cancer (eg. gliomas, astrocytomas, glioblastomas), melanoma, lymphoma, small bowel cancers (duodenal and jejunal), leukemia, pancreatic cancer, hepatobiliary tumours, germ cell cancers, prostate cancer, head and neck cancers, thyroid cancer or sarcomas, and wherein more preferably the subject is a human.
